(19) United States
(12) Patent Application Publication
(10) Pub. No.: US 2023/0055790 A1
(43) Pub. Date: Feb. 23, 2023

(54) MELT-EXTRUDABLE 3D PRINTING INKS

(71) Applicants: MASSACHUSETTS EYE AND EAR INFIRMARY, Boston, MA (US); PRESIDENT AND FELLOWS OF HARVARD COLLEGE, Cambridge, MA (US)

(72) Inventors: Nicole Leah BLACK, Cambridge, MA (US); Emily DAVIDSON, Cambridge, MA (US); Jennifer A. LEWIS, Cambridge, MA (US); Aaron Kyle REMENSCHNEIDER, Boston, MA (US); Elliott D. KOZIN, Boston, MA (US)

(21) Appl. No.: 17/784,346

(22) PCT Filed: Dec. 11, 2020
(86) PCT No.: PCT/US2020/064627

§ 371 Date: Jun. 10, 2022

Related U.S. Application Data
(60) Provisional application No. 62/947,389, filed on Dec. 12, 2019.

(51) Int. Cl. A61L 27/18; C08L 75/06

(52) U.S. Cl. CPC A61L 27/18; C08L 75/06; A61L 2430/14

(57) ABSTRACT

Described herein are melt-extrudable biodegradable inks for 3D-printing, methods of using the inks, and kits including the inks, to prepare implantable grafts, such as artificial tympanic membrane devices or artificial cartilage, nerve conduit, tendon, muscle tissue, or bone devices.

### CROSS-REFERENCE TO RELATED APPLICATIONS

This application claims priority to U.S. Patent Application Ser. No. 62/947,389, filed Dec. 12, 2019, which is incorporated herein by reference in its entirety.

### FEDERALLY SPONSORED RESEARCH OR DEVELOPMENT

This invention was made with Government support under Grant Nos. 2T32DC000038-26 and 4T32DC000038-25 awarded by the National Institutes of Health and DGE11144152 from the National Science Foundation. The Government has certain rights in the invention.

### TECHNICAL FIELD

The present document relates to methods, compositions, devices, and systems for the 3D printing of biomedical graft material.

### BACKGROUND

Three-dimensional (3D) printing is a type of additive manufacturing in which a desired 3D shape or object is built up from an available supply of material. In some cases, the material is initially a solid that is temporarily melted, a liquid that is solidified, or a powder that is solidified during the manufacturing process. Examples of 3D printing techniques include stereolithography, in which a photo-responsive resin is hardened with a laser; fused filament fabrication (FFF), in which a solid material is melted, printed, and fused to surrounding material when solidified; filamentary extrusion/direct ink writing, in which the ink is extruded from a nozzle head via pressure and the resultant object can be cured or sintered; and granular material binding, in which a bed of granular material is bound, often with heat or a fluid binder.

Other 3D additive manufacturing methods include Stereolithography (SLA), Digital Light Processing (DLP), Electron-beam melting (EBM), Selective laser melting (SLM), Selective heat sintering (SHS), Selective laser sintering (SLS), Direct metal laser sintering (DMLS), Laminated object manufacturing (LOM), and Electron Beam Freeform Fabrication (EBF3). 3D printing presents an opportunity to carefully program the architecture of biodegradable materials that encourage cell ingrowth, alignment, and remodeling along the print path.

### SUMMARY

Provided herein are melt-extrudable inks suitable for 3D printing which include biodegradable polymers, and in some instances fugitive porogenic materials. Also provided herein are methods of using the melt-extrudable inks and kits including the melt-extrudable inks.

In one aspect, the disclosure provides melt-extrudable biodegradable inks for 3D printing, the ink including: a soft segment block, and a hard segment block, wherein the molar ratio of soft segment block to hard segment block is in a range from 1:1.2 to 1:2.0.

In another aspect, the disclosure provides melt-extrudable biodegradable inks for 3D printing, the ink including: a biodegradable polymer and a fugitive porogen material, wherein the fugitive porogen material is present at a weight percent (wt %) of no more than 50 wt %.

In some embodiments of these inks, the biodegradable polymer includes a soft segment block and a hard segment block.

In some embodiments of these inks, the soft segment block includes one or more of polycaprolactone (PCL), poly(ethylene glycol) (PEG), poly(hexamethylene carbonate)

(PHC), poly(ethylene oxide) (PEO), poly(propylene oxide) (PPO), polylactide, (PLA), polyglycolide (PGA), poly(hydroxybutyrate) (P3HB and P4HB), poly(citric acid), poly(sebacic acid), amino acids, or other poly(ester), poly(ether), poly(carbonate), poly (tetramethylene oxide) (PTMO), poly(propylene fumarate) (PPF), and poly(amide) soft segments.

In some embodiments of these inks, the soft segment block is a diol formed from one or more of polycaprolactone (PCL), poly(ethylene glycol) (PEG), poly(hexamethylene carbonate) (PHC), poly(ethylene oxide) (PEO), poly(propylene oxide) (PPO), polylactide, (PLA), polyglycolide (PGA), poly(hydroxybutyrate) (P3HB and P4HB), poly(citric acid), poly(sebacic acid), amino acids, or other poly(ester), poly(ether), poly(carbonate), poly (tetramethylene oxide) (PTMO), poly(propylene fumarate) (PPF), and poly(amide) soft segments.

In some embodiments of these inks, the hard segment block includes one or more of isophorene diisocyanate (IPDI), methyl diphenyl diisocyanate (MDI), 1-lysine diisocyanate (LDI), 1,4-butane diisocyanate (BDI), hexamethylene diisocyanate (HDI), trimethylhexamethylene diisocyanate (TMDI), ethyl diisocyanate (ELDI), methyl diisocyanate (MLDI), and 1,4-cyclohexane diisocyanate (CHDI).

In some embodiments of these inks, the biodegradable polymer comprises one or more of hyaluronic acid (HA), poly(glycerol sebacate), poly(l,8-octanediol citrate), 0 poly(limonene thioether), poly (lactic-co-glycolic acid) (PLGA), polyurethane, poly(ester urethane)urea (PEUU), poly(carbonate urethane) urea (PECUU), collagen, fibrin, nylon, and silk.

In some embodiments of these inks, the molar ratio of soft segment block to hard segment block is 1:1.2 to 1:1.8.

In some embodiments of these inks, the molar ratio of soft segment block to hard segment block is 1:1.5.

In some embodiments of these inks, the soft segment and hard segment are present in a ratio needed to make a poly(ester urethane)urea (PEUU).

In some embodiments of these inks, the inks include a chain extender.

In some embodiments of these inks, wherein if a chain extender is present, the chain extender includes one or more of ethylene glycol, 1,4-butanediol, 1,4-cyclohexanedimethanol, diamines including 1,2-ethanediamine, 1,4-butanediamine, combinations including 2-amino-1-butanol, or other degradable linkages such as 2-hydroxyethyl-2-hydroxyproponoate.

In some embodiments of these inks, the inks include a fugitive porogen.

In some embodiments of these inks, wherein if a fugitive porogen is present, the fugitive porogen is an oligomer including poly(ethylene glycol) or poly(propylene glycol).

In some embodiments of these inks, wherein if a fugitive porogen is present, the fugitive porogen includes one or more of pluronic, alginate, gelatin, polyacrylic acid, poly(acrylate), poly(methacrylate), poly(maleic acid), poly(ethylene oxide), acrylates, methacrylates, water-soluble proteins, water-soluble polysaccarides, water soluble salts, or water-soluble small molecules such as sugars (for example, dextran).

In another aspect, this disclosure provides grafts including the melt-extrudable biodegradable inks for 3D printing described by any of the embodiments above.

In another aspect, this disclosure provides methods of fabricating grafts including the melt-extrudable biodegradable inks for 3D printing described by any of the embodiments above, the method including: melt-extruding the melt-extrudable biodegradable ink for 3D printing through a nozzle, e.g., a 10-1000 μm inner diameter nozzle, from a heated extrusion print head. In some instances, the nozzle has an inner diameter of 10, 25, 50, 75, 100, 125, 150, 175, 200, 225, 250, 300, 400, 500, 600, 700, 800, 900, or 1000 μm. In some embodiments, the nozzle has an inner diameter of 200 μm.

In yet another aspect, this disclosure provides methods of implanting grafts including the melt-extrudable biodegradable inks for 3D printing described by any of the embodiments above into a patient to heal or augment a tympanic membrane or to replace a missing tympanic membrane or missing portion thereof, the method including: accessing the damaged or missing tympanic membrane (e.g., through a bilayer design or handle on the graft); obtaining an appropriately sized and configured graft in the form of an artificial tympanic membrane device; and securing the artificial tympanic membrane device to seal the damaged portion of the tympanic membrane or replacing the missing tympanic membrane or missing portion thereof.

In some embodiments, the grafts including the melt-extrudable biodegradable inks for 3D printing described by any of the embodiments above further includes a cellular adhesion and/or a cell invasion-inducing material to promote tissue adhesion and cell growth.

In some embodiments, the grafts including the melt-extrudable biodegradable inks for 3D printing described by any of the embodiments promote cellular alignment and deposition of extracellular matrix proteins along the print path via anisotropic topographical, chemical, or mechanical properties present in the graft.

In yet another aspect, this disclosure provides methods of implanting grafts including the melt-extrudable biodegradable inks for 3D printing described by any of the embodiments above into a patient to heal or augment vasculature tissue, cartilage, a nerve conduit, a tendon, muscle tissue, or a bone or to replace a missing portion of vasculature tissue, cartilage, nerve conduit, tendon, muscle tissue, or bone, the method including: accessing the damaged or missing vasculature tissue, cartilage, nerve conduit, tendon, muscle tissue, or bone (e.g., through a bilayer design or handle on the graft); obtaining an appropriately sized and configured graft in the form of an artificial vasculature tissue, cartilage, nerve conduit, tendon, muscle tissue, or bone device; and securing the artificial cartilage, nerve conduit, tendon, muscle tissue, or bone device to seal the damaged portion of the vasculature tissue, cartilage, nerve conduit, tendon, muscle tissue, or bone, or to replace the missing portion of cartilage, nerve conduit, tendon, muscle tissue, or bone.

Any numerical range recited herein includes all sub-ranges subsumed therein. For example, a range of “1 to 10” includes all sub-ranges between and including the recited minimum value of 1 and the recited maximum value of 10, that is, having a minimum value equal to or greater than 1 and a maximum value equal to or less than 10.

As used herein, the terms “comprising,” “comprise,” and “comprised,” and variations thereof, are open-ended terms. The terms “a” and “an” mean one or more.

As used herein, the term “patient” or “subject” refers to members of the animal kingdom, including, but not limited to, mammals, including, but not limited to, humans.

As used herein, “biodegradable” materials are those that, when introduced into cells, are broken down by cellular machinery (e.g., enzymatic degradation) or by hydrolysis into components that cells can either reuse or dispose of without having toxic effects on the cells. Further, components generated by breakdown of a biodegradable material may not induce inflammation and/or may not cause local or systemic toxicity in vivo. Additionally, biodegradable polymeric materials break down into their component polymers. The breakdown of biodegradable materials (including, for example, biodegradable polymeric materials) includes hydrolysis of ester bonds. Breakdown of materials (including, for example, biodegradable polymeric materials) includes cleavage of urethane and urea linkages. These degradation processes can be enhanced by the presence of hydrolytic enzymes, including lipases, proteases, ureases, and/or esterases.

The methods and compositions described herein include the following benefits and advantages. First, the methods allow for rapid customization of the 3D printed tissue graft. Specifically, due to the nature of the new inks, and the melt-extrusion temperature being slightly above the melting temperature, 3D printed ink filaments solidify rapidly. Thus, structures can be printed at high aspect ratios (for example, up to 1:20 ratio between the base:height of the part) and high resolution.

Second, components and devices, e.g., graft devices and other implants, printed with the new inks can induce cellular alignment and/or deposition of extracellular matrix proteins (i.e. collagen, fibronectin, and/or laminin) along complex 3D print paths. Because the ink solidifies quickly after being printed, it may be that the polymer chains lock in their elongation created by shear forces in the nozzle. This causes grafts to be stiffer along the print path and cells to align, even on topographically flat surfaces.

Third, the presently described methods achieve relevant mechanical properties for soft tissue repairs and have good surgical handling. Materials produced by the methods described herein have a Young's modulus of 5-500 MPa, e.g., 10-400 mPa, 20-250 mPA, 30-100 MPa (tunable with PEG addition and print parameters), which is very close to living tissues. The printed devices also have good structural integrity, even when thin 50-100 μm devices such as grafts or implants are flexed and manipulated through small cavities.

Fourth, the new methods and devices also provide relevant in vivo degradation rates and tissue adhesion/integration. The degradation half-life of the printed devices can be between about 1 and 12 months, e.g., about 1-6 months, or about 1-3 months, depending on the material and the specific use. For example, a shorter degradation half-life may be preferable to longer degradation half-lives of implant materials that are not intended to remain permanently in the body. Additionally, the materials promote cellular in-growth and angiogenesis—likely through the urethane and urea bonds that resemble peptide bonds for cells to form formal adhesions.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### DESCRIPTION OF DRAWINGS

FIG. 1 is a series of schematics showing ideal graft properties, which include surgical feasibility, acoustic properties, customization/versatility, adhesion to the TM, biodegradation, and cellular alignment.

FIG. 2 is an illustration showing the various tissue grafts that may benefit from our technology.

FIG. 3A shows conical TM grafts utilizing stereolithography 3D-printed substrates.

FIG. 3B shows bilayer TM grafts designs that can enable one-handed transcanal placement of grafts for in-clinic TM repair.

FIG. 3C shows biomimetic grafts for TMJ cartilage discs.

FIG. 3D shows biodegradable grafts for placing over a normal TM to passively enhance sound conduction by strengthening radial stiffness.

FIG. 4A is a series of schematics showing the two-step synthesis of biodegradable poly(ester urethane urea) (PEUU). In the first step, a polycaprolactone (PCL) soft segment is reacted with a 1,4-diisocyanatobutane (BDI) hard segment to create poly(ester urethane). Then, in the second step, 1,4-diaminobutane (BDA) is used to extend the chains and to impart urea bonds in the final polymer.

FIG. 4B is a series of schematics that show that introduction of a fugitive porogen increases water uptake of grafts for enhanced degradation and tissue adhesion

FIG. 5 is a series of schematics that show the synthesis of the inks created for 3D printing of biomimetic TM grafts. (Top row) Schematic views of pure PCL and porous P-PCL inks and (bottom row) Schematic views of pure PEUU and porous P-PEUU inks blended with 25 wt % PEG. PEG is removed by immersing the 3D-printed grafts in water.

FIGS. 6A-6B are schematic illustrations of TM graft fabrication via high operating temperature-direct ink write (HOT-DIW).

FIGS. 7A-7B are schematic illustrations of tensile specimens 3D printed from PCL, P-PCL, PEUU, and P-PEUU inks.

FIGS. 8A-8B are graphs showing the characterization of PEUU and composite P-PEUU (PEUU+25 wt % PEG) material.

FIG. 9 is a graph showing apparent viscosity as a function of shear rate for thermoplastic biodegradable inks.

FIGS. 10A-10D are graphs showing filament width of 3D printed lines of 4 biodegradable inks from 200 μm inner diameter nozzles.

FIGS. 11A-11G are a series of graphs showing 3D printing of 8 mm biomimetic 50C/50R TM grafts by HOT-DIW. The scale bars represent 2 mm.

FIGS. 12A-12C are graphs showing the validation of PEG leaching from 8 mm biomimetic 50C/50R TM grafts via FTIR, mass loss, and PBS absorption.

FIGS. 13A-13D are graphs showing the mass and in vitro degradation profile of 8 mm biomimetic 50C/50R TM grafts (n=6).

FIGS. 14A-14B are graphs showing the proliferation of two common cell types responsible for TM remodeling on 8 mm biomimetic 50C/50R TM grafts (n=6).

FIGS. 15A-15B are graphs showing the mechanical properties of tensile specimens (n=6).

FIGS. 16A-16F are images showing the alignment of GFP-HNDFs and collagen I deposition on P-PEUU grafts printed at a speed of 20 mm/s.

FIGS. 17A-17G are optical microscopy images (FIG. 17A) and graphs (FIGS. 17B-17G) showing 3D printing of square grafts to determine the impact of material print speed, and nozzle diameter on cellular alignment after 7 days of GFP-HNDFs culture on the topographically flat surface of grafts (n=6).

FIGS. 18A-18C are schematics (FIG. 18A) and images (FIGS. 18B-18C) showing HOT-DIW of biomimetic circular/radial P-PEUU grafts result in alignment of collagen I along the print path.

FIGS. 19A-19C are schematics that show common mechanisms of tympanic membrane perforations and that current tympanic membrane graft materials do not effectively restore tympanic membrane structure and function. FIG. 19A shows tympanic membrane perforations can be caused by a variety of mechanisms. FIG. 19B shows intact human tympanic membrane (top) and damaged, i.e., perforated human tympanic membrane (bottom). FIG. 19C shows currently utilized autologous graft materials have mismatched and inconsistent mechanical properties, causing poor healing outcomes (left) and that these isotropic graft materials do not have the circular and radial collagen fiber arrangement of the tympanic membrane, leading to poor hearing outcomes (right).

FIGS. 20A-20H show the creation and repair of chronic subtotal perforations. FIG. 20A shows that an incision was made behind the bulla in a chinchilla, which exposed exposing the middle ear space, as shown in FIG. 20B. FIG. 20C shows a normal chinchilla TM. FIG. 20D shows chronic perforations were created using a thermal myringotomy loop. FIG. 20E shows that these chronic perforations persisted for 1 month without spontaneous healing. Underlay tympanoplasty was performed with (FIG. 20F) autologous fascia grafts, (FIG. 20G) Biodesign® grafts, and (FIG. 20H) biomimetic P-PEUU 50C/50R grafts with a diameter of 8 mm.

FIG. 21A is a series of images of a human TM and various 3D-printed P-PEUU grafts, as well as two standard graft materials, including fascia control porcine SIS and an laser Doppler vibrometry (LDV) graph that demonstrates superior velocity for 3D-printed 50C/50R grafts, particularly at high frequencies.

FIG. 21B is a series of images of a human TM and various 3D-printed P-PEUU grafts as well as two standard graft materials, and corresponding thermal images that demonstrate complex motion patterns at high frequencies, similar to the human tympanic membrane.

FIG. 22A is a series of photos that show 3D-printed and melted forms of the same grafts to form isotropic control grafts in the same thickness.

FIG. 22B is a laser Doppler vibrometry (LDV) graph that demonstrates superior velocity for 3D-printed 50C/50R grafts, even compared to the thinnest isotropic melted grafts. Melted versions of 50C/50R grafts have a poor response, showing the importance of anisotropic architecture for sound conduction.

FIG. 22C is a series of digital opto-electronic holography (DOEH) images that show that anisotropic architecture is important to sound conduction and that melted grafts do not perform as well.

FIG. 23A is a photo that shows a tympanic membrane graft with 25 circular and 25 radial fibers that was generated using a 200 μm nozzle, and was melt-extruded at 90° C., 30 psi, 15 mm/s.

FIG. 23B is a photo that shows a bottom circular surface of a TM graft that demonstrates how GFP-labeled human neonatal dermal fibroblasts can align along the complex circular print path.

FIG. 23C is a photo that shows a top radial surface of a TM graft that demonstrates how GFP-labeled human neonatal dermal fibroblasts can align along the complex radial print paths.

FIGS. 24A-24I are a series of endoscopic images showing the repair and healing outcomes following underlay tympanoplasty of chronic subtotal perforations in chinchilla models. Endoscopic images of underlay tympanoplasty with (FIG. 24A) temporalis fascia graft, (FIG. 24B) Biodesign® graft, and (FIG. 24C) 3D-printed P-PEUU 50C/50R graft. Healed TMs 3 months post-implantation are endoscopically viewed for (FIG. 24D) temporalis fascia graft, (FIG. 24E) Biodesign® graft, and (FIG. 24F) 3D-printed P-PEUU 50C/50R graft. The number of failed grafts and healed grafts for (FIG. 24G) temporalis fascia grafts, (FIG. 24H) Biodesign® grafts, and (FIG. 24I) 3D-printed P-PEUU 50C/50R grafts.

FIGS. 25A-25F are histological sections of celloidin-fixed temporal bones showing the cross-section of the TM following underlay tympanoplasty of chronic subtotal perforations in chinchilla models with various graft materials. Hematoxylin and eosin (H&E) staining with light microscopy. Top row shows 1.25× magnification of TMs containing (FIG. 25A) healed fascia grafts, (FIG. 25B) healed Biodesign® grafts, (FIG. 25C) P-PEUU 50C/50R grafts. The location of the remodeled TM is indicated by black dashed boxes. Bottom row shows 20× magnification of TMs containing (FIG. 25D) healed fascia grafts, (FIG. 25E) healed Biodesign® grafts, (FIG. 25F) P-PEUU 50C/50R grafts. The left side of each image is the lateral side adjacent to the external auditory canal (EAC), while the right side of images is the medial side adjacent to the middle ear space.

FIGS. 26A-26B are bar graphs showing the hearing threshold changes (initial hearing thresholds minus 3 month post-tympanoplasty hearing thresholds) following underlay tympanoplasty of chronic subtotal perforations in chinchilla models with various graft materials. FIG. 26A shows hearing thresholds detected by distortion product otoacoustic emissions (DPOAE) and FIG. 26B shows hearing thresholds detected by auditory brainstem response (ABR). Values shown are mean with error bars representing±SD. (*p<0.05 from one material group, #p<0.05 from both material groups). Higher values closer to 0 indicate hearing restoration closer to normal and therefore improved tympanoplasty hearing outcomes.

FIGS. 27A-27F are histological sections of the cochlea showing ototoxic effects following underlay tympanoplasty of chronic subtotal perforations in chinchilla models. Hematoxylin and eosin (H&E) staining with light microscopy of cochlear sections. The top row shows the organ of Corti visualized at 20× magnification following tympanoplasty with (FIG. 27A) healed fascia grafts, (FIG. 27B) healed Biodesign® grafts, (FIG. 27C) P-PEUU 50C/50R grafts. The bottom row shows the modiolus of the cochlea showing position of spiral ganglion neurons (SGN) following tympanoplasty with (FIG. 27D) healed fascia grafts, (FIG. 27E) healed Biodesign® grafts, (FIG. 27F) P-PEUU 50C/50R grafts. The locations of these features are designated by black arrows.

FIG. 28 is a schematic that shows a tympanic membrane perforation and placement of an autologous tissue graft, such as fascia, that does not remodel. Credit: Shawna Snyder.

FIG. 29 is a schematic that shows a tympanic membrane perforation and placement of a biomimetic tympanic membrane graft on the medial side of the perforation. The graft degrades and remodels into tissue in a biomimetic architecture. Credit: Shawna Snyder.

FIG. 30 is a schematic that shows placement of a biomimetic tympanic membrane graft on the medial side of an intact TM to augment the structure of the tympanic membrane. The graft degrades and remodels into tissue in a biomimetic architecture, strengthening and building the tissue in the graft architecture. Credit: Shawna Snyder.

FIG. 31 is a schematic that shows placement of bilayer tympanic membrane grafts through the ear canal, enabling in-clinic placement. Credit: Shawna Snyder.

FIG. 32 is a schematic that shows placement of a single layer tympanic membrane graft through the ear canal and through a hole in the tympanic membrane, either via a perforation to heal the perforation, or via an incision made by the surgeon or clinician to enable augmentation of an intact tympanic membrane. Credit: Shawna Snyder.

### DETAILED DESCRIPTION

Soft tissue damage is common among individuals subjected to blast and traumatic injuries. Many soft tissues, such as muscle, nerve, articular cartilage, and collagenous tissues exhibit a complex anisotropic structure (different properties in multiple directions). One example is the tympanic membrane (TM), commonly known as the eardrum, which has a complex circular/radial microstructure and conical macrostructure. The TM is the most commonly damaged organ during blasts encountered by civilians and military personnel. TM perforation results in hearing loss, ear infections, ear pain, and dizziness. Repair of the eardrum is a complex procedure with a high rate of failure and poor hearing outcomes.

For the TM specifically, the Young's Modulus of the native human TM has been reported to range from 20 to 90 MPa depending on the direction and portion being tested and method used. Matching the mechanical properties of soft tissue grafts is crucial for appropriate host integration and to mitigate potential effects such as weakening of grafts that are softer than the host tissue or stress shielding and retraction of grafts that are stiffer than the host tissue. While elastomeric materials such as poly(dimethyl siloxane) have been 3D printed with success, these materials do not degrade, leaving sites for host infections in the body and often creating foreign body responses and thick scar tissue around the implant. Thus, the use biodegradable materials rather than permanent materials are ideal for most regenerative and tissue engineering applications. Most commonly used biodegradable polymers for 3D printing are orders of magnitude stiffer than soft tissues, such as PCL (300-400 MPa) and PLA (3,600 MPa). These materials can be difficult to manipulate through the small cavities in the ear without fracturing and can have trouble integrating with the surrounding tissue. Other materials commonly used as extracellular matrix mimics in bioprinting applications include gelatin or fibrinogen (1-100 kPa), and these materials are orders of magnitude softer and more ductile than tissues. These soft materials can also re-perforate or dislodge easily from the perforation.

Certain issues arise with current biodegradable polymers for 3D printing of soft tissue grafts. For example, the current biodegradable polymers for 3D printing are orders of magnitude stiffer than soft tissues, which are usually between 1-100 MPa. They additionally have poor adhesion to surrounding tissue, causing grafts to detach or retract from the remnant tissue, causing the tissue defect to re-emerge. Further, the current biodegradable polymers for 3D printing have a very slow degradation rate, causing grafts to become thicker as tissue grows on them, and creating foreign sites for infection and negative immune responses. Additionally certain current polymer inks require toxic solvents that must be evaporated to solidify the ink, which leaves toxic residues behind.

Polyurethanes are elastomeric materials composed of macrodiol soft segments connected by urethane bonds to diisocyanate groups of hard segments. These polymers can be synthesized to be biodegradable through the use of monomers that form hydrolytically and enzymatically degradable bonds, such as ester, ether, urethane, and urea bonds. Traditional synthesis protocols for biodegradable polyurethanes yield thermoset polymers that are processed by dissolving the polymer in a solvent or synthesizing the polymer at the time/site of device fabrication. This disclosure provides synthesis protocols that yield a thermoplastic biodegradable polyurethane with shear thinning rheology that can be extruded via heated direct ink write 3D printing without the need for any solvents in the printing process.

Devices, e.g., grafts or implants, 3D printed using the new inks described herein can be made to have a nanoporous structure by incorporating a fugitive water-soluble porogen, e.g., a water-soluble polymer such as polyethylene glycol (PEG), into the ink and leaching it out of a device after printing. This structure allows printed devices to absorb more water or other bodily fluids, increasing the degradation rate, helping graft devices to adhere better to remnant tissue, and increasing nutrient diffusion through the graft devices to cells. The porous structure also enhances the diffusion of degradation products away from the structure, which in turn also improves the degradation rate.

In addition, when hot melt extruding the material through small-diameter nozzles, the resultant polymer is stiffer along the print path, which induces fibroblasts and other cells to adhere to the printed devices in alignment with the print direction likely via mechano-transduction, even on topographically flat and confluent printed grafts. Thus, the graft devices are remodeled into extracellular matrix (e.g., collagen fibrils) and tissues that resemble the original print path when the polymeric material of the device degrades over time.

The disclosure provides new polymeric inks for 3D printing. The new inks are composed of custom-synthesized thermoplastic biodegradable polymers that can be mixed with fugitive porogen materials (e.g., water-soluble polymers, salts, proteins, sugars, polysaccharides, and fibers). The inks can be extruded via hot melt direct ink writing into custom architectures. Following printing, the porogen can be leached from the grafts, leaving the biodegradable polyurethane graft with interconnected, nanoscale pores in the graft. The grafts can be plasma treated for increased sterility and hydrophilicity, enabling better cell adhesion. Prior to implantation, the grafts can be soaked in PBS, growth factors, protein solutions, drug solutions, media, or more to absorb these fluids and retain them within their structure for increasing tissue adhesion, cell growth, or disease treatment.

### I. Melt-Extrudable, Biodegradable Inks for 3D Printing

Provided herein are melt-extrudable biodegradable inks, which comprise biodegradable polymers comprising a soft segment and a hard segment. A chain extender can also be optionally present.

### Soft Segment Block

“Soft segment blocks” (also referred to herein as “soft segment(s)”) are usually a polyether or polyester polyol that provide elasticity to the end-product. Suitable non-limiting examples of soft segment blocks include: diols formed from polycaprolactone (PCL), poly(ethylene glycol) (PEG), poly(hexamethylene carbonate) (PHC), poly(ethylene oxide) (PEO), poly(propylene oxide) (PPO), polylactide, (PLA), polyglycolide (PGA), poly(hydroxybutyrate) (P3HB and P4HB), poly(citric acid), poly(sebacic acid), peptides, or other poly(ester), poly(ether), poly(carbonate), and/or poly(amide) soft segments can also be used.

### Hard Segment Block

“Hard segment blocks” (also referred to herein as “hard segment(s)”) are usually composed of a diisocyanate and contribute strength and rigidity through physical cross-linking points to the end-product. Suitable non-limiting hard segment blocks include: isophorene diisocyanate (IPDI), methyl diphenyl diisocyanate (MDI), 1-lysine diisocyanate (LDI), 1,4-butane diisocyanate (BDI), hexamethylene diisocyanate (HDI), or trimethylhexamethylene diisocyanate (TMDI), or any other diisocyanate.

### Ratio of Soft Segment Block: Hard Segment Block

The molar (or end group) ratio of soft segment to hard segment is in a range of 1:1.2 to 1:1.8. The ratio of hard segment depends upon the hydrogen bonding present in the hard segment block. In other words, if a given polymer has more or higher hydrogen-bonding, greater amounts of this polymer increases the difficulty in melting the polymer. As such, hard segment blocks having high hydrogen-bonding are required at a lower ratio compared to hard segment blocks having lower hydrogen-bonding. For example, BDI has high crystallinity (i.e., high hydrogen-bonding), so a ratio of 1:1.5 to 1:1.6 would be ideal. For IPDI, which has low crystallinity (i.e., low hydrogen-bonding), a ratio range of 1:1.6 to 1:1.8 would be ideal.

The ratio of soft to hard segment can be determined by one of ordinary skill in the art depending on the chosen hard segment block and the degree of hydrogen-bonding in the hard segment block. Some hard segment blocks are aromatic-ring-containing structures and have high crystallinity due to pi stacking between rings. However, since their byproducts are benzene-like, they are not ideal for biomedical applications. This too would be appreciated and readily apparent to one of skill in the art.

### Chain Extenders

A chain extender can optionally be included to extend chains and to impart urea bonds into the end-product, to make a stiffer, tougher, and more biocompatible material. Suitable chain extenders include, but are not limited to: ethylene glycol, 1,4-butanediol, 1,4-cyclohexanedimethanol, diamines including 1,2-ethanediamine, 1,4-butanediamine, combinations including 2-amino-1-butanol, or other degradable linkages such as 2-hydroxyethyl-2-hydroxyproponoate. When the chain extender is a diol (ethylene glycol, butanediol, the dimethanol species) it creates urethane bonds, while when the chain extender is a diamine it creates urea bonds (therefore, the hard/soft segment block that is chain extended must have diisocyanate end groups). When the chain extender is present, the molar ratio of soft segment:hard segment:chain extender is in a range of 1:1.2:0.6 to 1:1.8:0.9.

### II. Porogens for Melt-Extrudable Biodegradable Inks for 3D Printing

Also provided herein are melt-extrudable biodegradable inks comprising a fugitive porogen material melt-blended with the biodegradable polymer.

### Fugitive Porogen Material

“Fugitive porogen” or “fugitive porogenic material,” as referred to herein, is any material used to make pores in molded structures, e.g., used for tissue engineering. More specifically, the porogen is present during the formation of a material or device, e.g., a graft or implant material used for the tissue engineering, and the porogen is then subsequently removed from the material or device. The removal of the porogen leaves pores, e.g., nanopores or micron scale pores, within the material or device. These pores are ideal for tissue-engineered grafts as they enable infiltration fluid absorption by the graft, increasing the hydrolytic degradation rate and enhancing nutrient transport within the graft.

Suitable fugitive porogens for the melt-extrudable biodegradable inks for 3D printing as described herein include polymeric porogens, which are described for instance in “Effect of Porogens (Type and Amount) on Polymer Porosity: A Review” (S Mane, Canadian Chemical Transactions, 2016, Volume 4, Issue 2, Pages 210-225), which is incorporated herein by reference in its entirety. Briefly, oligomers including one or more of poly(ethylene glycol) or poly(propylene glycol) with different molecular weights can be used for the fabrication of the melt-extrudable inks for 3D printing.

Other suitable fugitive porogens include: water-soluble polymers (ex: pluronic, alginate, gelatin, polyacrylic acids, poly(acrylates), poly(methacrylates), poly(maleic acid)); water-soluble salts (e.g., NaCl, K₂Cr₂O₇, CaCl₂); water-soluble proteins (e.g., uncrosslinked collagen, fibrin); sugars (e.g., glucose, fructose, galactose); or water-soluble polysaccharides and fiber (e.g., pullulan, psyllium).

The fugitive porogen is present at a weight percent in a range of 10 wt % to 50 wt %.

### Biodegradable Polymer Component

The fugitive porogen is combined, e.g., melt-blended or solvent-blended, with a suitable biodegradable polymer component. In the case of solvent-blending, the solvent can be evaporated from the ink prior to printing. In one instance, the fugitive porogen is combined with the biodegradable polymer as described above in Section I. Specifically, the fugitive porogen can be combined with a biodegradable polymer comprising a hard segment and a soft segment and optionally a chain extender block. Suitable non-limiting examples of soft segments, hard segments, and chain extenders include those listed above.

In other instances where the biodegradable polymer does not comprise a hard segment, a soft segment, and optionally a chain extender block, the biodegradable polymer component is hyaluronic acid (HA), poly(glycerol sebacate), poly(1,8-octanediol citrate), poly(limonene thioether), polyurethane, poly(ester urethane)urea (PEUU), poly(carbonate urethane) urea (PECUU), poly(lactic-co-glycolic acid), collagen, cellulose, fibrin, nylon, silk, poly(caprolactone), poly(lactic acid), or poly(glycolic acid).

### III. Properties of the Melt-Extrudable, Biodegradable Inks for 3D Printing

The graft devices produced from the melt-extrudable biodegradable inks can further include one or more of a cellular adhesion and/or a cell invasion-inducing material, e.g., growth factors. The graft devices can further include one or more cells, e.g., fibroblasts, chondrocytes, keratinocytes, stem cells, progenitor cells, neurons, myoblasts, endothelial cells, and epithelial cells. The cells can be harvested from the patient or from different sources, e.g., a transplant from another subject or from cultured cell lines. The growth factors can include a fibroblast growth factor (FGF), epidermal growth factor (EGF), vascular endothelial growth factor (VEGF), platelet-derived growth factor (PDGF), and a keratinocyte growth factor (KGF). These growth factors can be included either directly in the entire infill or preferentially patterned during the 3D printing process to replicate native growth factor gradients or polarize sides of the tympanic membrane (TM) to promote and “tune” ingrowth of different cell types. The devices can further include one or more drug eluting materials.

In various embodiments, the graft devices can have a diameter of 0.5 to 50 millimeters, e.g., 1, 2, 3, 5, 7, 9, 10, 15, 20, 25, 30, 35, 40, 45, 50, or any range in between. The graft devices can have a diameter based on a specific patient, e.g., a human patient. The graft devices can have a thickness of 10 to 750 microns, e.g., 25, 50, 75, 100, 125, 150, 175, 200, 250, 300, 400, 500, 600, or 750 microns. In some embodiments, the graft devices are impermeable to air while in other embodiments they can be permeable to air. The graft devices can also be designed to be permeable to one or more drugs or other agents including small molecules, biologics, steroids, and antibiotics.

In some embodiments, the graft devices can include an ossicular connector on one surface of a tympanic membrane graft. The ossicular connector can be formed as an artificial umbo, malleus, or stapes and take the shape of one of an umbo, malleus, or stapes, or of a ring, a hinge, loop, archway, or a ball or socket, or some combination thereof. For example, such ossicular connectors can be secured to a surface of an artificial tympanic membrane graft devices, e.g., an underlay graft device. In various embodiments, the connector can connect to a remnant ossicular chain in the patient's middle ear or to an ossicular prosthesis implanted in the middle ear before or at the same time as the tympanic membrane graft(s) are implanted.

In some embodiments, the graft can include features the enable two or more grafts to be combined at the time of surgery. For example, a “lock-and-key” mechanism would allow a perforation to be sandwiched between two grafts. These features could enable the facile and rapid placement of grafts in-office via transcanal endoscopic techniques. Other 3D features, such as conical architectures, can also be included in the grafts. In some embodiments, the graft can be placed adjacent to intact tissue to heal or augment its function. For example, a radially stiff graft could be placed laterally on an intact tympanic membrane, allowing the native tissue to regenerate stronger fibers in the radial direction. Thus, sound conduction to the ossicles could be enhanced. Additionally, cartilage, vasculature (e.g., blood vessels or a portion of a blood vessel), nerves (e.g., nerve conduit), tendons, muscle tissue, bone, temporomandibular joint discs, hernia, or cardiac, including heart valves could be strengthened by implanting grafts of the material around or adjacent to the native tissue.

Important properties for the ideal membrane graft have been identified (those specific to the tympanic membrane are noted below. Thickness for grafts range from 10 μm to 2 cm and suitable ranges for width/length of grafts include:
Tympanic membrane grafts (including both TM repair and augmentation): 1 mm to 20 mm
Temporomandibular joint discs: 1 mm to 20 mm
Vascular grafts: 100 μm to 10 cm
Muscle grafts: 100 μm to 10 cm
Nerve conduits: 100 μm to 100cm
Cartilage grafts, including sinonasal repair grafts and articular knee cartilage grafts: 100 μm to 10 cm
Hernia grafts: 100 μm to 10 cm
Cardiac grafts, including heart valves: 100 μm to 100 mm

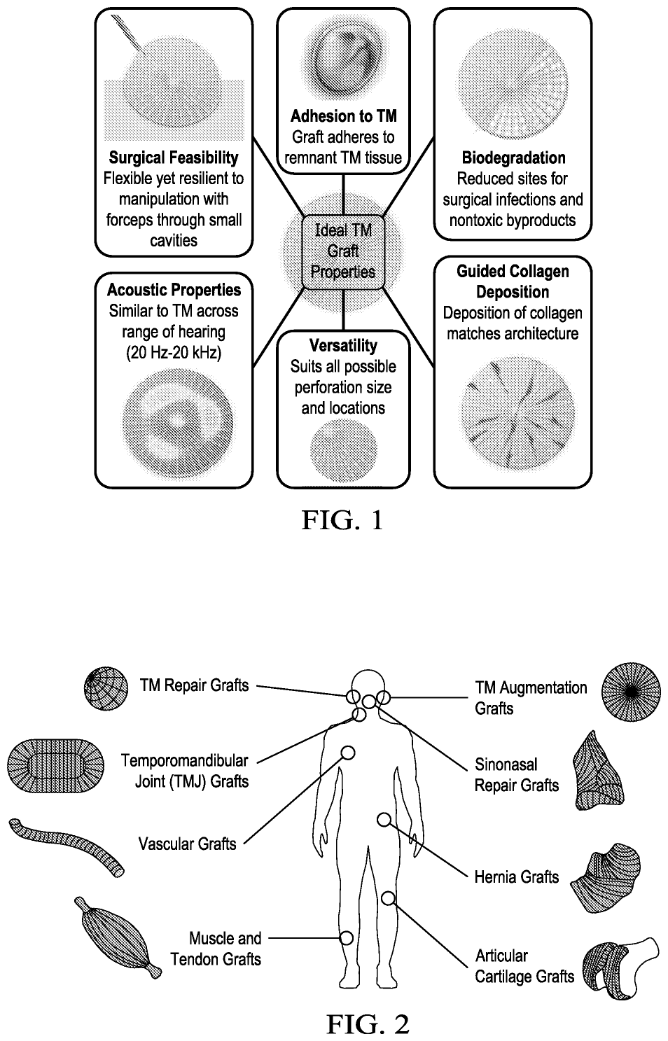

Several important properties for the ideal tympanic membrane graft have been identified (FIG. 1, see also Table 1 below). Some of these properties relate to the choice of material itself, such as surgical feasibility, adhesion to the TM, and biodegradation. Other properties relate to both the material and the manufacturing method, such as acoustic properties, customization, and the potential for cellular alignment.

TABLE 1
Ideal resultant features of the inks described
herein (specific for tympanic membrane grafts)

| | Criteria | Design Goals |
|---|---|---|
| Material | Surgical | Graft thickness from 80-120 μm |
| Design | Feasibility | Ability to retain its shape after |
| | | flexing |
| | Adhesion to | Young's Modulus between |
| | the TM | 10-100 MPa |
| | | Failure of grafts (i.e. graft |
| | | retraction) is lower than fascia |
| | Biodegradation | Complete graft degradation |
| | | achieved in 2-6 months in vivo |
| Material and | Acoustic | Ability to conduct sound to the |
| Manufacturing | Properties | ossicular chain from 20 Hz-20 kHz |
| Design | Versatility | Grafts can be altered to match the |
| | | perforation size and location |
| | Guided Collagen | Ingrowing cells remodel the graft |
| | Deposition | into an anisotropic collagen |
| | | structure |

Desired structural and handling properties of the graft devices can slightly vary depending on the intended end-use. For instance, for tympanic membrane grafts, the graft should be made from a biodegradable material that can be resorbed by the body as the patient's cells lay down native tissue. This can reduce sites for infections and foreign body reactions. Biodegradability also enables the remodeled tympanic membrane to return to its original 80-120 micrometer (μm) thickness. As the graft is flexed through small cavities, it should be elastomeric so as to return to its original geometry without additional manipulation. After placement, the material must adhere to the remnant tympanic membrane and have similar mechanical properties to the native tympanic membrane to prevent retraction. Then, as the graft is remodeling with native tissue, it should guide collagen deposition into a circular and radial collagen architecture to allow the tympanic membrane to vibrate well at both low and high frequencies. The manufacturing method also allows for customization of the grafts to match the region of the tympanic membrane that is perforated so that the remodeled structure can best match this region. For ideal ENT experience in placement, healing outcomes, and hearing outcomes, the ideal TM graft would consist of a biodegradable, elastomeric material with easy handling properties and matched circumferential and radial architecture of the native TM with potential to remodel into this architecture.

The degradation byproducts of the graft should also be systemically nontoxic. Additionally, ototoxic efforts of small molecule byproducts of the graft must be avoided. Ototoxic effects have been observed for small molecule therapeutics and have been shown to create irreversible sensorineural hearing loss.

Additional features of the TM grafts, include hydrophilicity and porosity. TM grafts should be able to absorb fluids and growth factors, enabling a suitable environment for native cells to grow onto the graft and incorporate with the remnant TM tissue on the medial and lateral surfaces.

Alongside the material, the manufacturing method by which TM grafts are produced could be critical to their function. There are several techniques to encourage anisotropy in engineered tissue, including mechanical stimulation, electrical stimulation, micropatterning, cellular confinement between topographical features, electrospinning, and rotary jet spinning. Many of these methods have limited complexity or can be challenging to apply during an in vivo environment. Cellular alignment and migration correspond highly to the alignment of extracellular matrix proteins that contribute to the mechanical functions of the tissue. Fibrillar collagen (type I, II, and II collagen) is formed when procollagen is secreted into the extracellular space parallel to the direction of highest spreading. These procollagen molecules are cleaved together to form a triple-helical collagen molecule. If a cell is elongated, collagen fibers will be formed in the direction of spreading. Thus, controlling the cell alignment will in turn control collagen fiber alignment in the tissue. By utilizing mechanotransduction, cells can sense the mechanical properties of their environment. Inside a cell, integrin-based adhesion complexes couple the actin cytoskeleton of a cell to its substrate. Cells generate larger cytoskeletal forces on stiff substrates than on soft substrates, as demonstrated by the Hill Curve. Thus, cells on stiffer substrates spread out more than those on soft substrates, which have a rounded morphology.

Other ideal features of TM graft also relate to the material but can also be enabled by unique manufacturing mechanisms, such as 3D printing. As previously described, the hearing outcomes of patients across the standard range of human hearing, 20 Hz-20 kHz, are heavily reliant upon the structure of graft placed. Desired features of the melt-extrudable biodegradable inks for 3D printing include ability to hold form/flexibility/foldability, stiffness (e.g., a Young's Modulus of less than 100 MPa), an optimal degradation rate (e.g., less than 6 months, anywhere between days and less than 6 months, 2 weeks-5 months, 1 month-3 months, etc.), degradation in water, adhesion to surrounding tissue (e.g., sufficient adhesion to allow grafts to attach to surrounding remnant tissue thereby preventing perforation or tissue defect to re-emerge). Additionally, hearing outcomes of patients across the standard range of human hearing, 20 Hz-20 kHz, are heavily reliant upon the structure of graft placed. While maintaining a graft with Young's Modulus (E) in the 10-100 MPa range of the normal human TM will help, those in the lower range (e.g. E=30 MPa “soft” material) will have an improved ability to match impedance of air at low frequencies but not vibrate well at high frequencies, while those materials in the upper range (e.g. E=100 MPa “soft” material) will be able to drive the umbo but will struggle to vibrate at low frequencies Thus, it is necessary to develop a TM graft that will perform well at both low and high frequencies. One method to do so is to mimic the native TM structure through the inclusion of a circular and radial fibrous architecture. This may enable its vibration as one sheet of a “soft” material at lower frequencies, with the radial fibers enabling more complex modes of motion for the graft to behave as a “stiff” material at higher frequencies.

TM perforations occur in multiple locations and in multiple sizes. Thus, the macroscale size and microscale fiber arrangement of the missing TM region vary widely between patients. Thus, there are two important aspects of TM grafts to ensure versatility. The first is in the macroscale size and shape of the graft. While grafts do not necessarily need to be tuned for a specific patient, they should be able to be tuned to match a range of sizes of the perforations. If a 3D printing approach is taken, rapid customization is indeed feasible. However, most TM perforations are in a standard set of sizes and locations: Grade 1 (0-1-25% of the TM, 0-2.5 mm in diameter), Grade II (26-50% of the TM, 2.5-5 mm in diamater), Grade III (51-75%, 5-7.5 mm in diameter), and Grade III (76-100% 7.5-10 mm in diameter). Thus, either creating a kit of various graft sizes to suit most perforations or having the ability to readily cut a graft material into the desired size would also likely enable perforation closure. Similarly, the second major aspect, which is matching the fiber arrangement of the missing location, could also be enabled either by subtracting material from a larger graft and orienting the graft during placement. While TM graft customization is important, TM grafts do not necessarily need to be 3D printed for a specific patient, as long as the chosen graft suits the perforation size and fiber arrangement.

### IV. Methods of Using the Melt-Extrudable Biodegradable Inks for 3D Printing

The disclosure features methods of implanting grafts made from melt-extrudable biodegradable inks into a patient in need thereof.

The melt-extrudable biodegradable inks for 3D printing described herein can be used in many different applications, and in particular are beneficial for situations where anisotropy is required in the graft material. Examples include, tympanic membrane, articular cartilage grafts, arterial grafts, heart valves, skeletal muscle grafts, smooth muscle grafts, and nerve grafts (see, e.g., FIG. 2). Other soft tissues without anisotropy, such as facial plastic implants and reconstructive grafts, could benefit from the 3D printed aspects of the technology, as this could enable patient-specific customization.

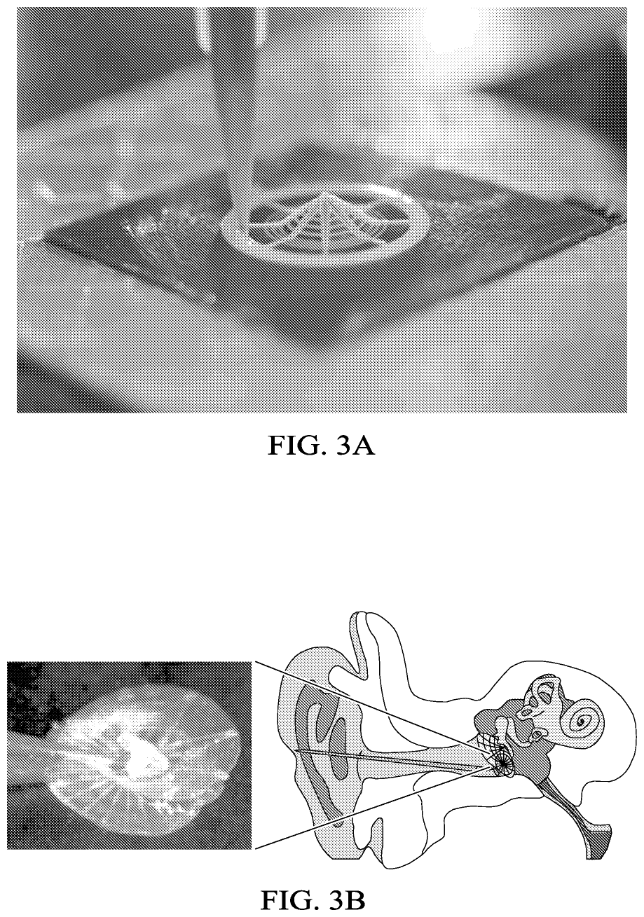

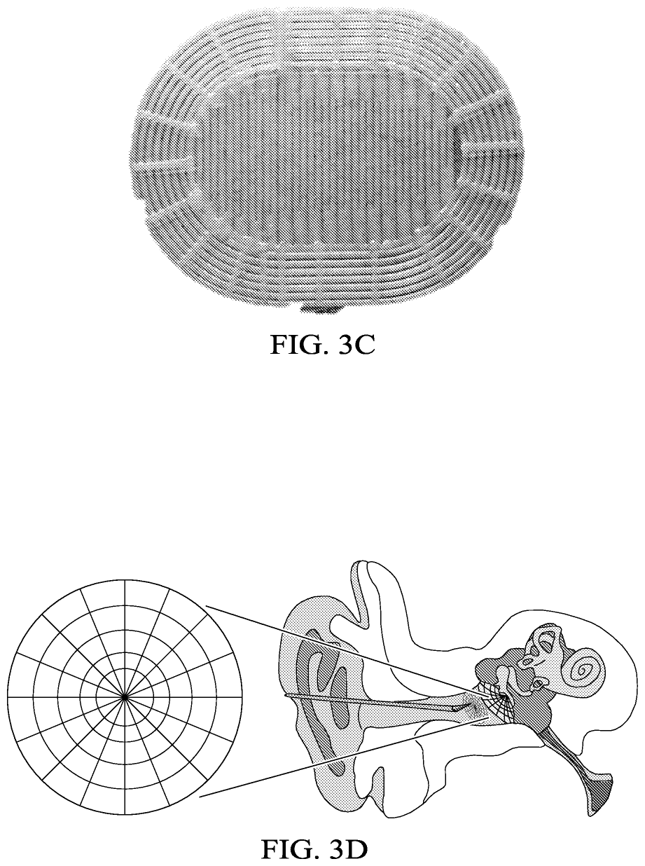

Articular cartilage is a type of hyaline cartilage containing oriented collagen II fibers. One example is the articular cartilage disc in the temporomandibular joint (TMJ). In the central region of the disc, collagen fibers predominately run anterioposteriorly. Around the periphery, there is an interwoven ring-like structure, wherein the anterioposterior band fibers run predominately mediolaterally with some interwoven anterioposterior directed fibers. The central region is significantly stiffer than medial and lateral regions and the mediolaterally, posterior region is significantly stiffer than central and anterior regions. Thus, TMJ disc stiffness, indicated by Young's Modulus and Instantaneous Modulus, was higher in directions corresponding to high fiber alignment. TMJ grafts 3D-printed using the inks described herein in an architecture that mimics this can be achieved (see, e.g., FIGS. 3A-3D).

Vascular grafts can also be enabled by this technology. In the medial layer of an artery wall, collagen fibers are arranged in two helically distributed arrangements. These fibers are aligned in the circumferential direction with very little deviation. In the adventitial and intimal layers, the orientation of the collagen fibers is dispersed. The dispersion of the orientation of collagen fibers in the adventitia of human iliac arteries has a significant effect on their mechanical response. In addition, the alignment of smooth muscle cells and vascular endothelial cells in the inner layers of arteries are oriented perpendicular to the direction of flow, like the inner collagen fibers. This orientation is crucial for guiding the direction of blood flow.

Besides the alignment of collagen fibers, the anisotropic and direction elongation of cells themselves can have a significant impact on function of soft tissues. This is particularly true for muscle tissue, where cells rely on this alignment for proper communication with each other. In skeletal muscle, tubular muscle cells called myofibers comprise tubular myofibrils with multiple nuclei. Myofibrils containing repeating sections of sarcomeres, which are long, fibrous protein filaments that slide past each other which a muscle contracts or relaxes. These muscle fibers can take on a variety of shapes and orientations to perform the motion of interest. The shear wave speed and therefore resultant force is highly dependent upon the direction of the muscle fibers. Thus, if one desired to graft muscle tissue, it would be ideal to encourage cells to regrow along the alignment of the missing portion of the muscle tissue. This also includes grafts for hernia repair.

Nerve tissue is composed of neurons, which receive and transmit impulses, along with glial cells which help the propagation of these impulses. Neurons have long axons that send action potential signals to the next cell. Therefore, the positioning and alignment of these cells relative to each other is crucial for propagation of the signal in the intended direction. Since nerve grafting is a very sensitive process that requires precise alignment, nerve grafts are usually autologous nerve tissue collected from elsewhere in the patient. However, this requires an additional surgical site and damage to the host nerve for the tissue. This invention could enable researchers to fabricate aligned neuronal grafts in vitro using explant cultures from the patient's cells and implant them once they have matured.

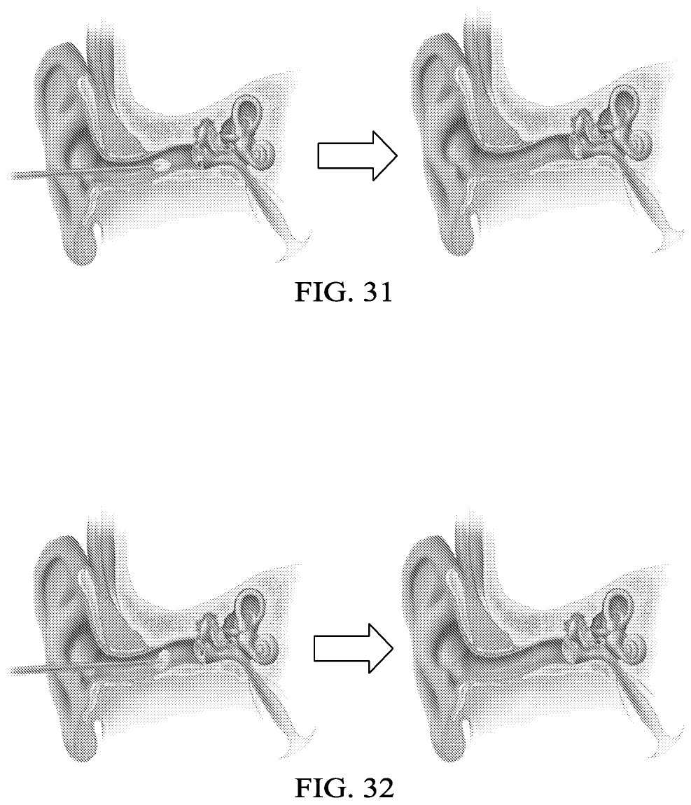

The 3D printing inks described herein can be used to make artificial tympanic membrane grafts for a patient to heal or augment a damaged tympanic membrane or to replace a missing tympanic membrane or portion thereof, e.g., to repair a perforation. FIG. 30 shows an example of a graft being used to augment the structure of an intact tympanic membrane. FIG. 31 shows placement of bilayer tympanic membrane grafts through the ear canal, enabling in-clinic placement. FIG. 32 shows placement of a single layer tympanic membrane graft through the ear canal and through a hole in the tympanic membrane, either via a perforation to heal the perforation, or via an incision made by the surgeon or clinician to enable augmentation of an intact tympanic membrane. The disclosure also features the use of any of the devices described herein to heal, augment, or replace a damaged or missing tympanic membrane. The methods include accessing the damaged or missing tympanic membrane; obtaining an appropriately sized and configured artificial tympanic membrane device; and securing the artificial tympanic membrane device to seal the damaged portion of the tympanic membrane or replacing the missing tympanic membrane or missing portion thereof.

### V. Methods of Making Melt-Extrudable Biodegradable Inks for 3D Printing

The disclosure also features methods of fabricating melt-extrudable biodegradable inks for 3D printing.

One method includes the following steps:

1. Drying a soft segment block,

2. Reacting the soft segment block with a hard segment block,

3. Optionally adding a chain extender to the reaction, and

4. Precipitating and drying the resultant polymer.

Another exemplary method includes mixing a biodegradable polymer (such as a polyurethane) with a fugitive porogen. In this method, prior to using the 3D printed graft, the graft is soaked in water to leach out the fugitive porogen (see e.g., FIG. 5).

### VI. Kits for Using the Melt-Extrudable Biodegradable Inks for 3D Printing

Generally, also provided herein are kits comprising the melt-extrudable biodegradable inks for 3D printing described herein. In one embodiment, the kit comprises a composition comprising a hard segment, soft segment, chain extender and a fugitive porogenic material. In one embodiment, the kit comprises a biodegradable polymer (such as a polyurethane or biodegradable polymers without block segment) and a fugitive porogenic material.

The kits described above also optionally comprise one or both of a mold for molding (casting, etc.) the bioscaffold into a shape. The kit, in some instances, is equipped with a variety of scaffold sizes such that an option for any perforation is readily available.

The components of the kits described above are packaged in any packaging suitable for shipping and storage of the components of the kit, such as, for example: boxes, containers, bottles, vials, test tubes, plastic wrap, foil, etc. as are apparent to one of ordinary skill.

### EXAMPLES

The invention is further described in the following examples, which do not limit the scope of the invention described in the claims.

The following Materials and Methods apply to the Examples that follow.

### Materials and Methods

### Synthesizing the Meltable Biodegradable Polyurethane

A thermoplastic biodegradable polyurethane was synthesized composed of soft and hard segments and different chain extender ratios, whose composition was optimized over several iterations by assessing its ability to be extruded by high operating temperature-direct ink writing (HOT-DIW) (Table 2, below).

Synthesis of the biodegradable polyurethane was done through a two-step reaction to create poly(ester urethane urea) (PEUU) where the soft segment was polycaprolactone diol (PCL, Mₙ=2000, Sigma-Aldrich) with hydrolysable ester bonds, the hard segment was butane diisocyanate (BDI, Sigma-Aldrich), and the chain extender was 1,4-butanediamine (BDA, Sigma-Aldrich). These were combined in a molar ratio of 1:1.5:0.75 soft segment : hard segment : chain extender. First, the PCL was dried in a vacuum oven at 100° C. overnight to remove the residual water before synthesis.

The dried PCL was added to a 3-neck flask along with 30 wt % dimethyl sulfoxide (DMSO, Sigma-Aldrich) under the flow of nitrogen at 70° C. Then, BDI was added to the flask along with 0.01 wt % stannous octoate catalyst (Spectrum Chemical). This first step formed the urethane bonds in the polymer.

After 2 hours, the BDA was mixed in additional DMSO to bring the solution to 20 wt % polymer in solvent. The reaction continued for 1 more hour under nitrogen. This extended the chains with urea bonds, making it stiffer and more biocompatible, as the urea bonds resemble peptide bonds to cells. After the reaction was complete, it was precipitated in water to remove DMSO and dried in a vacuum oven at 100° C. to obtain the final polymer.

TABLE 2
Synthesis protocols showing molar ratios trialed between
soft segment (PCL-diol), hard segment (BDI), and chain
extender (BDA) to achieve melt-processable polymer.

| Soft | Hard | Chain | |
|---|---|---|---|
| Segment | Segment | Extender | |
| PCL-diol | BDI | BDA | Notes |
| 1 | 2 | 1 | Inaccessible Tₘ |
| 1 | 1.75 | 1 | Inaccessible Tₘ |
| 1 | 1.7 | 1 | Inaccessible Tₘ |
| 1 | 1.6 | 1 | Inaccessible Tₘ |
| 1 | 1.6 | 0.8 | Inaccessible Tₘ |
| 1 | 1.5 | 1 | Thermoplastic, Tₘ ≈ 190° C. |
| 1 | 1.5 | 0.75 | Thermoplastic, Tₘ ≈ 40° C. |

Creation of Inks with Fugitive PEG

To create composite inks with a fugitive component, PEUU and PCL (control) (Mn=50,000, Sigma-Aldrich, USA) were combined with 25 wt % PEG (Mn=1500, Sigma-Aldrich, USA). To facilitate mixing, PEUU was dissolved in acetone at 75 wt/vol % (VWR International, USA) and PCL was dissolved toluene at 75 wt/vol % (Reagent Grade, Ricca Chemical, USA). The solutions were heated at 50° C. for PEUU/PEG/acetone inks and 100° C. for PCL/PEG/toluene inks for 1 hours before blending in a high-speed mixer (FlackTek, USA) at 2000 rpm for 5 min. Following complete mixing, the solvents were evaporated in a vacuum oven at 60° C. for PEUU/PEG/acetone inks and 120° C. for PCL/PEG/toluene inks for 24 h. PEUU and PCL inks were also produced without PEG (porogen) following the same solution and evaporation processes. Four inks were therefore generated for characterization: PCL, P-PCL (PCL+25 wt % PEG1500), PEUU, and P-PEUU (PEUU+25 wt % PEG1500) (FIG. 5) and, ultimately, printing biomimetic TM grants.

### Biomimetic TM Graft Fabrication Via 3D Printing

A custom 3D multi-material printer (Aerotech) with a ˜1 μm resolution was equipped with a custom-designed hot printhead from which the ink was extruded pneumatically. A custom Aerobasic G-code program was used to control the print path, height, speed, and extrusion temperature. This method, known as HOT-DIW, was used to melt extrude the PEUU and PCL-based inks at elevated temperatures. The printhead contains a machined copper block containing a custom-machined steel 3 mL syringe coupled to a high-pressure adaptor (HPx High-Pressure Dispensing Tool, Nordson EFD, USA), with an enclosing fluoroplastic insulating block. Two 100-W 0.25″×2″ cartridge heaters (Omega Engineering, USA) were controlled via a resistance temperature detector sensor adjacent to the syringe. Feedback control was provided via a PID Controller (Platinum Series Versatile High Performance PID Controllers, Omega Engineering, USA).

To create consistent graft architecture from each ink, the print parameters were optimized such that extruded ink filaments (100 μm in width and 50 μm in height) were achieved. The inks were extruded through tapered-tip stainless steel nozzles (inner diameter=200 μm, Tecdia Inc., Japan) held at 90° C. for P-PCL and P-PEUU or 115° C. for PCL and PEUU. A custom Aerobasic G-code program was designed to create a meandering path that changes extrusion pressure with each line. Four print speeds of 5, 10, 15, and 20 mm/s were tested at extrusion pressures ranging from 40 to 100 psi. For each combination of ink, print speed, and extrusion pressure, 6 lines of length of 5 mm were printed onto a glass slide substrate. The width of each fiber was measured using a Keyence digital microscope (VHX2000, Keyence, Japan) and recorded to determine print parameters required to obtain filaments with the desired width and height.

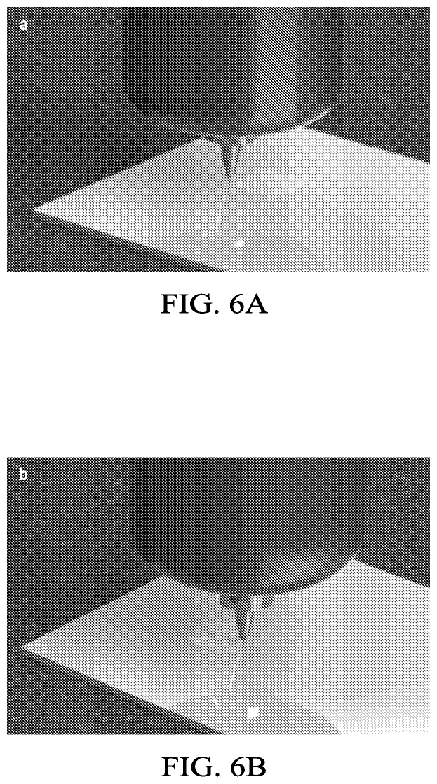

For in vitro cellular alignment studies, square grafts (8 mm×8 mm) were printed using both PEUU- and PCL-based inks at a speed of 20 mm/s (Error! Reference source not found. 6A). To investigate print speed effects on cellular alignment, additional grafts were fabricated at print speeds of 5, 10, and 15 mm/s using the same 200 μm nozzle. To create isotropic P-PEUU grafts, the printed grafts (200 μm nozzle diameter at 20 mm/s speed) were melted to “erase” any anisotropy imparted by HOT-DIW. For in vitro degradation and cell proliferation studies, biomimetic TM grafts composed of PCL, P-PCL, PEUU, and P-PEUU with an overall diameter of 8 mm were printed in a circular and radial architecture (FIG. 6B). A series of 50 concentric circles were printed first, spaced 80 μm apart at 20 mm/s. Next, a series of 50 radial lines from the center of the grafts were printed at 20 mm/s. The resultant biomimetic TM grafts were defined by their 50 concentric circular (C) followed by 50 radial (R) (50C/50R) structure.

Leaching of Fugitive PEG from Grafts

3D-printed grafts were soaked in DI water at 37° C. to remove PEG from the grafts. At each timepoint, the grafts were removed from the DI water, dried with a Kim wipe, and then dried in an oven at 100° C. overnight. Masses were taken before and after leaching to ensure complete removal of the PEG phase from the composite material, creating the interconnected porous network.

### Differential Scanning Calorimetry

The melting behavior of PEUU and P-PEUU inks was measured by differential scanning calorimetry (DSC) (Q200 calorimeter, TA Instruments, USA). Samples of PEUU and P-PEUU (prior to PEG leaching) were hermetically sealing inside aluminum pans (TZero, TA Instruments, USA). Samples were analyzed via a heat-cool-heat cycle between −50° and 200° C. at a rate of 10° C./min to clear the thermal history of the material. The melting temperature, Tm, was determined from the summit of the melting peak.

### Rheological Characterization

The rheological properties of each ink were measured using a controlled-stress rheometer (Discovery HR-3 Hybrid Rheometer; TA Instruments, USA) equipped with a 20 mm peltier plate geometry. To simulate printing conditions during HOT-DIW, a temperature sweep was performed at a temperature of 90° C. for P-PCL and P-PEUU inks and at 115° C. for PCL and PEUU inks, after holding for 5 min to equilibrate at the desired temperature. Viscometry measurements were carried out by subjecting the inks to an increasing shear rate swept from 0.01-100 s⁻¹ at 1 Hz. All 4 inks (PCL, P-PCL, PEUU, and P-PEUU) were analyzed for Newtonian or shear thinning rheological properties. Printing temperatures were chosen so that 100 μm filaments can be successfully extruded from 200 μm inner diameter (ID) nozzles, which was conducted because the inks cannot be extruded through smaller ID nozzles using the current HOT-DIW setup.

### TM Graft Post-Processing and Sterilization

All printed grafts were placed in a plasma treatment system (Diener Femto PCCE, Germany) and exposed to oxygen plasma for 30 sec to both render their surfaces hydrophilic and to achieve sterilization. The grafts were then placed under UV germicidal irradiation in a biosafety cabinet for 5 minutes per top and bottom surface. To determine the impact of sterilization on molecular weight of the polymer, PEUU grafts sterilized via plasma and UV germicidal radiation also underwent standard ethylene oxide (EtO) processing at a temperature of 30° C. The total EtO exposure time was 16 hours (4 hours for injection +12 hours of holding), followed by 3 hours purge and 1 hour aeration.

To remove PEG, biomimetic TM grafts (50C/50R) were immersed in deionized (DI) water at 37° C. thereby producing P-PCL and P-PEEU grafts. This process was also carried out on biomimetic TM grafts (50C/50R) printed using pure PCL and PEUU inks to determine if any mass loss arises that cannot be attributed to PEG leaching. The minimum time required for complete PEG leaching was determined by placing the grafts in DI water at 37° C. and removing them at predetermined timepoints of 0.16, 0.5, 1, 2, 4, 8, 24, and 48 hours (n=3 for each graft per timepoint). Excess water was removed from the grafts using KimWipes (Kimberly-Clark, USA) following by drying in a vacuum oven at 37° C. overnight (AT09.110.UL, Across International, USA). Grafts were weighed before and after soaking at each timepoint. Thermogravimetric analysis (TGA) (TA Instruments, USA) was used to weigh the grafts with accuracy of 0.0001 mg.

Upon leaching PEG from each TM graft, samples (n=6 of each graft) were placed in 1X Dulbelco's phosphate buffered saline (PBS) without calcium and magnesium (Corning, USA) for 30 min. The grafts were weighed before and after this process to determine the amount of PBS absorbed by each graft (i.e., PCL, P-PCL, PEUU, and P-PEUU grafts).

### Fourier-Transform Infrared Spectroscopy

To assess the presence of urethane and urea bonds in PEUU as well as PEG incorporation within these inks, Fourier-transform infrared spectroscopy (FTIR) in double-sided transmittance mode (Bruker Hyperion Vertex 70 FTIR Spectrometer, Bruker Corporation, USA) was performed on biomimetic 50C/50R P-PEUU and PEUU grafts after printing and post-leaching for varying immersion times. Their initial and final (post-leaching) IR spectra were compared to ensure complete removal of PEG.

### Cell Viability

The proliferation of human keratinocytes and fibroblasts on biomimetic TM grafts was studied, including an assessment of cell alignment and collagen deposition. Primary human epidermal keratinocytes (HEKs) (ATCC, USA) were cultured in dermal cell basal medium (ATCC, USA) supplemented with keratinocyte growth kit (ATCC, USA) according to the manufacturer's instructions. Cells were kept in an incubator (VWR International, USA) at 37° C. with 5% CO₂ atmosphere up to passage 10. Cell medium was pre-warmed and replaced every 2 days. At 80% confluency, cells were passaged with 0.05% trypsin-0.53 mM EDTA (ATCC, USA), which was deactivated with trypsin neutralizing solution (ATCC, USA). Human neonatal dermal fibroblasts modified with enhanced green fluorescent protein (GFP, λₑₓ=488 nm), (GFP-HNDFs, Angio-Proteomie, USA) were cultured in Dulbecco's modified eagle medium supplemented with 862 mg·L⁻¹ L-alanyl-glutamine (Gibco, USA) and 10% fetal bovine serum (FBS, ATCC, Manassas, Va.) in an incubator at 37° C. with 5% CO₂ atmosphere. Cell medium was pre-warmed and replaced every 2 days up to passage 20. At 80% confluency, cells were passaged with 0.05% trypsin-0.53 mM EDTA (Gibco, USA). For cell proliferation assays, HEKs and GFP-HNDFs (100,000 cells per graft) were seeded onto the top surface of biomimetic TM grafts (8 mm 50C/50R grafts of PCL, P-PCL, PEUU, and P-PEUU) in a 48-well plate (n=6 per graft material). After allowing 1 hour for cell adhesion, additional media was placed in each well. At time points of 1, 3, 7, 14, 21, and 28 days, an MTS tetrazolium colorimetric assay (ab197010, Abcam, Cambridge, UK) was performed to assess cell proliferation. Each graft was moved into a new well to ensure that only cells growing on the grafts were included and to exclude cells growing on cell culture plastic of the well plates. 10 mL of assay buffer was mixed with 10 μl of bis-AAF-R110 substrate and stored at −20° C. between assays. A volume corresponding to 10% of the culture volume of sterile MTS in phosphate buffer solution was added to each culture well and incubated for 1 h at 37° C. with 5% CO₂ atmosphere. The solutions were transferred to a 96-well microwell plate and measured using absorbance at 490 nm on a plate reader (Synergy HT, BioTek Instruments, USA) to assess the relative cell proliferation on each graft material. Media was aspirated and replaced for the corresponding cell type after each assay (every 2-3 days). The average and standard deviation of absorbance values were calculated. A Student's t-test (IMP Pro 15, USA) was conducted to determine statistical significance between groups.

### Degradation Rate

In vitro degradation rates for printed biomimetic TM grafts (50C/50R) composed of each ink (PCL, P-PCL, PEUU, and P-PEUU) were measured. Prior to testing, each graft was weighed to obtain its initial mass (Mo). A lipase concentration of 100 U/mL to mimic biological conditions was used, as well as a lower lipase concentration of 1 U/mL. The tests were performed in both phosphate buffered saline (PBS, Lonza, USA) and in PBS containing a lipase concentration of 1 U/mL or 100 U/mL (Aspergillus Oryzae, Sigma, USA). The grafts were placed in a 24-well plate with 1 mL of solution per well. The samples were then placed on a rocker (LSE Platform Rocker, Corning, USA) at 60 rpm in an incubator at 37° C. with 5% CO₂ atmosphere. The grafts were removed from the solution at specific time points and rinsed using deionized water. The samples were dried at 37° C. in a vacuum oven and then weighed (Mₓ). The remaining graft mass was calculated using:

$$Percent = \frac{{M}_{x}}{{M}_{0}} * 100$$

Following sample weighing, the samples were placed back in the well plates, which were replenished with fresh PBS or lipase solutions.

### Mechanical Characterization

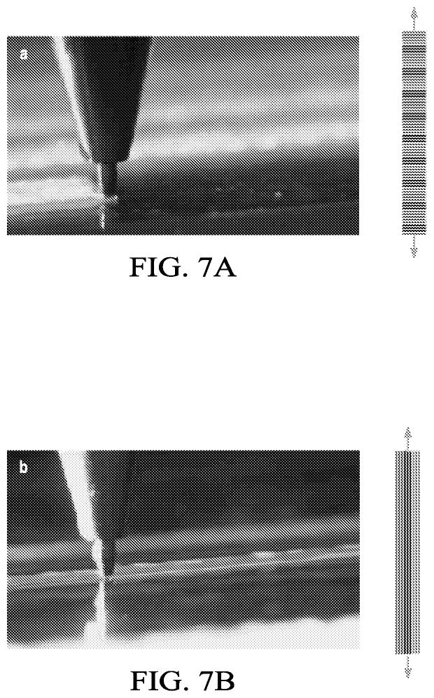

The elastic properties of the biomimetic TM grafts were measured under ambient conditions using tensile testing. Tensile specimens (48.0 mm long, 3.52 mm wide, and 100 μm thick) of each material (PCL, P-PCL, PEUU, and P-PEUU) were printed with filamentary features that were aligned either parallel and orthogonal to the direction of applied stress (FIGS. 7A-7B; the long axis and tensile direction was either (FIG. 7A) orthogonal to the print path or (FIG. 7B) parallel to the print path; all samples were printed utilizing a 200 μm inner diameter nozzle and a print speed of 20 mm/s; arrows indicate the direction of applied tension). Note, two layers were printed on top of one another to mimic the ˜100 μm thickness of the human TM in the pars tensa (Hawkins, J. E. (1976). Drug ototoxicity. In Auditory system (pp. 707-748). Springer, Berlin, Heidelberg).

An Instron machine (Model 3342, Norwood, Mass.) with a load cell maximum of 50 N was used to apply unidirectional load on specimens. Tabs were fabricated at the ends of 3D printed specimens using double-sided tape to prevent slippage from the tensile grips, resulting in a gage length (L₀) of 25 mm and width of 3.52 mm. Specimens were tested at a strain rate of 5 mm/s in a tensile direction both parallel and orthogonal to the print path for all 4 inks (n=6 each). Young's modulus (E) was calculated as the stress (σ) over strain (ϵ) at a displacement of 1 mm (4% of total gage length), as given by:

$$E = \frac{σ}{ε} = \frac{\frac{{F}_{n}}{A}}{\frac{Δ L}{{L}_{0}}} \qquad ( 5 )$$

where Fₙ was normal force acting orthogonal to the cross-sectional area (A) and ΔL/L was the strain. The mean value and standard deviation (SD) of Young's modulus were calculated for each graft (n=6 of each material for both print paths) and values parallel (E_{∥}) and orthogonal (E_{⊥}) to the uniaxial testing direction were reported. A Student's t-test (JMP Pro 15, Cary, N.C.) was conducted to determine statistical significance between E_{∥} and E₁₉₅ of each of the 4 inks (PCL, P-PCL, PEUU, and P-PEUU).

### In Vitro Fibroblast Alignment on TM Grafts

To characterize fibroblast alignment, GFP-HNDFs (100,000 cells per graft) were seeded onto the top surface of P-PEUU 20 mm/s square grafts as well as the bottom surface of square grafts printed with varying materials composition at 20 mm/s print speed (PCL, P-PCL, PEUU, and P-PEUU) and varying print speed of P-PEUU ink (5, 10, 15, and 20 mm/s) as previously described (8 mm×8 mm, n=6 each). Each graft contained printed filaments (100 μm wide with a center-to-center spacing of 80 μm). Samples were fixed at day 7 (BD Cytofix™, BD Biosciences, US). Grafts underwent immunostaining for collagen I (1:500, rabbit monoclonal, ab138492, Abcam, USA). An upright confocal microscope (LSM710, Zeiss, Germany) equipped with a 5× objective was used for imaging. To eliminate edge effects, images were only acquired from the center-most 6×6 mm region. 3D projections and Z-stacks were generated using manual and automated processes in Imaris (Oxford Instruments, UK). The Directionality plugin through ImageJ (National Institutes of Health, US) was used to analyze the alignment of both GFP-fibroblasts and collagen I for each graft. Averages and standard deviations for found for each group from −90° to 90° across the graft, whereby 0° was the direction of the print path.

### In Vitro Acoustic Testing

3D-printed grafts along with control collagen sheets and cadaveric TMs were mounted onto a custom holder to fixate the graft or tissue. Digital Opto-Electronic

Holography (DOEH) was performed by previously established techniques (Cheng J T, Aarnisalo A A, Harrington E, del Socorro Hernandez-Montes M, Furlong C, Merchant S N, Rosowski J J. Motion of the surface of the human tympanic membrane measured with stroboscopic holography. Hearing research. 2010 May 1;263(1-2):66-77) at four different frequencies across the human range of sound perception: 400, 1000, 3000 and 6000 Hz. Laser Doppler vibrometry (LDV) was also conducted by previously established techniques (Aarnisalo A A, Cheng J T, Ravicz M E, Hulli N, Harrington E J, Hernandez-Montes M S, Furlong C, Merchant S N, Rosowski J J. Middle ear mechanics of cartilage tympanoplasty evaluated by laser holography and vibrometry. Otology & neurotology: official publication of the American Otological Society, American Neurotology Society [and] European Academy of Otology and Neurotology. 2009 Dec;30(8):1209) to determine the sound-induced velocity in the center of the grafts. Preliminary results demonstrated organized motion patterns in circular/radial PEUU grafts that increase in number with higher frequency, similar to the human TM. Additionally, preliminary LDV results showed that increasing the radial lines on 3D-printed PEUU grafts increases the velocity of the grafts at high frequencies.

### Chronic Perforation Creation

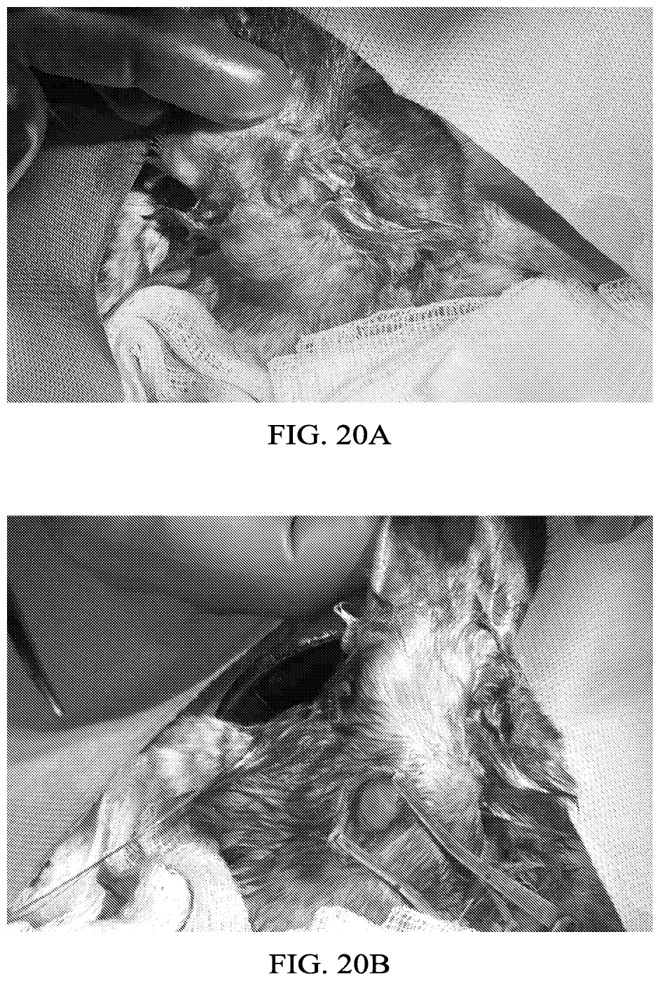

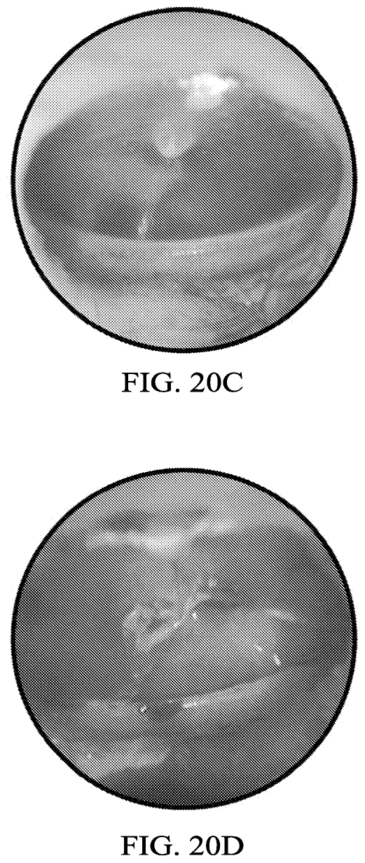

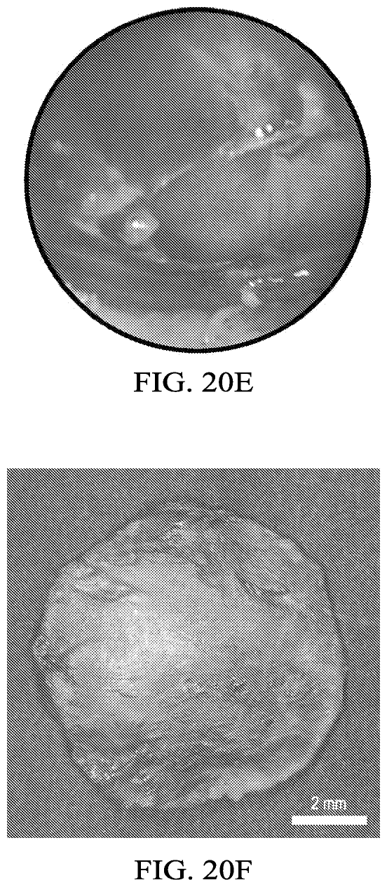

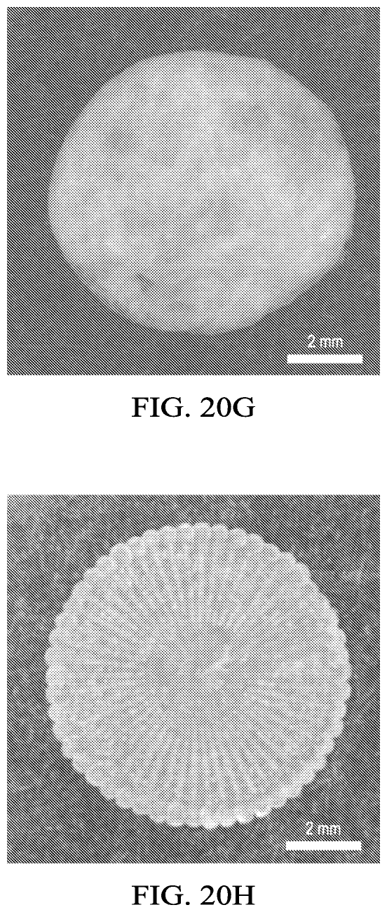

Lanigera chinchillas (˜500 grams) were anesthetized and monitored. Animals undergo baseline ABR and DPOAE testing in a sound treated booth as described in the below section. For control materials, fascia grafts were harvested from the superficial surface of the masticator muscle on chinchillas with a radius of approximately 8 mm. Biodesign® grafts were cut with a radius of approximately 8 mm as a second control material. Following hearing testing, the TM was visualized via using a rigid 0° and 30° Storz Hopkins® rod endoscope with a light source and camera (KARL STORZ, Germany). A low-temperature thermal myringotomy loop (Bovie Medical, USA) was used to create a 50% perforation on the inferior portion of the pars tensa (Amoils CP, Jackler RK, Milczuk H, Kelly K E, Cao K. An animal model of chronic tympanic membrane perforation. Otolaryngology—Head and Neck Surgery. 1992 Jan;106(1):47-55). The inner mucosal layer of the TM was removed using a 1 mm hook, and radial orientated incisions were made in the remnant TM. This process allows for infolding of epithelialized flaps. An identical procedure was then repeated on the contralateral ear. Weekly endo-otoscopy (weeks 1-4) was performed to ensure perforations remain stable and free of infection (FIGS. 20A-20E).

The animals undergo transcanal endoscopic tympanoplasty utilizing biomimetic P-PEUU 50C/50R TM grafts in one ear (n=20) with control autologous fascia grafts (n=8) or Biodesign® grafts (n=12) in the contralateral ear (FIGS. 20F-20H). Grafts were placed in an underlay, transbullar fashion. Following graft placement, the middle ear was packed with absorbable Gelfoam® (Pfizer, USA) for graft stabilization during healing. Antibiotic ofloxacin drops were applied to both ears daily starting 1 week following the procedure. All treated and control ears undergo regular otoscopic evaluation to determine healing rates. After 3 months, a binary decision was made as to whether the tympanoplasty was successful in closing the perforation based on endoscopic examination.

### Tympanoplasty

TM grafts were employed for implantation into a chronic TM perforation animal model via an IRB approved protocol to evaluate the stability of repair, wound healing, and otologic safety profile of materials. Control fascia grafts were harvested from the superficial surface of the masticator muscle. The animals then underwent transcanal endoscopic tympanoplasty utilizing 3D-printed PEUU TM grafts in one ear and fascia grafts in the contralateral ear. Grafts were placed in an underlay, transperforation fashion with middle ear gelfoam stabilization. Ofloxacin drops were applied to both ears daily starting 1 week following the procedure. All treated and control ears underwent serial otoscopic evaluation to determine healing rates.

### Auditory Testing

Animals undergo baseline threshold DPOAE and ABR measurements in a sound treated booth in both ears under general anesthesia. Standard procedures were used for conducting these measurements (Rosowski, J. J., Dobrev, I., Khaleghi, M., Lu, W., Cheng, J. T., Harrington, E., & Furlong, C. (2013). Measurements of three-dimensional shape and sound-induced motion of the chinchilla tympanic membrane. Hearing research, 301, 44-52; Slama, M. C., Ravicz, M. E., & Rosowski, J. J. (2010). Middle ear function and cochlear input impedance in chinchilla. The Journal of the Acoustical Society of America, 127(3), 1397-1410). For DPOAE measurements, pure tones were delivered in pairs, and the otoacoustic emissions were recorded. Measurements were obtained at 500, 1000, 2000, 4000, 8000, and 16,000 Hz starting at 10 dB and advancing by 5 dB steps. ABR thresholds were obtained first by using a click stimulus starting at 20 dB SPL and progressing by 5 dB steps until a clear ABR wave V response was observed at three sequential runs. Tone bursts were delivered, and electrodes measure activity from the auditory pathway. Pure tone ABR were obtained at 300, 1000, 2000, 4000, 8000, and 16,000 Hz starting at 10 dB and advancing by 5 dB steps. These electrical responses were analyzed, and the recordings were obtained in six to seven waveforms.

Hearing improvement following TM repair was determined by comparing the data before perforation (day 0) to post-perforation and 3 months following tympanoplasty. The mean and standard deviation (SD) of ABR and DPOAE threshold changes were calculated for each control and biomimetic group, using only data from successfully healed TMs to mitigate the pressure differential effect of residual perforations on non-healed TMs. A Student's t-test (JMP Pro 15, USA) was conducted to determine statistical significance of ABR and DPOAE threshold changes for each frequency between materials. A threshold difference greater than 10 dB was considered clinically significant. Statistical significance was defined as p<0.05.

### Histopathology

The chinchillas were sacrificed following hearing tests at approximately 3 months after the tympanoplasty procedure. Animals were perfused with 10% formalin through cardiac catheterization. Their temporal bones were harvested for histopathologic processing. The techniques for fixation, dehydration and embedding of ear tissues in celloidin were well described (Schuknecht H F, Merchant S N, Nadol J B. Schuknecht's pathology of the ear. People's Medical Pub. House-USA, Shelton. 2010). Decalcification of the skulls was performed with ethylenediaminetetraacetic acid (EDTA) over the course of 9 months. They were embedded in celloidin for sectioning. Celloidin provides a high level of anatomic detail over the entire auditory periphery and has demonstrated success in the preservation of the organ of Corti, enabling hair cell counts (Quesnel A M, Nakajima H H, Rosowski J J, Hansen M R, Gantz B J, Nadol Jr J B. Delayed loss of hearing after hearing preservation cochlear implantation: human temporal bone pathology and implications for etiology. Hearing research. 2016 Mar. 1; 333:225-34; Nadol Jr J B, Burgess B J, Gantz B J, Coker N J, Ketten D R, Kos I, Roland Jr J T, Shiao J Y, Eddington D K, Montandon P, Shallop J K. Histopathology of cochlear implants in humans. Annals of Otology, Rhinology & Laryngology. 2001 September; 110(9):883-91). Histologic sections demonstrating reconstructed TMs and controls were analyzed using H&E staining and light microscopy. P-PEUU degradation was assessed by measuring thickness and presence of the remnant graft in comparison to pre-implanted dimensions. Established techniques permit evaluation of the ototoxicity of P-PEUU material through hair cell and neuronal counts (Liberman MC, Kiang NY. Acoustic trauma in cats: cochlear pathology and auditory-nerve activity. Acta oto-laryngologica. 1978; Sugawara M, Corfas G, Liberman MC. Influence of supporting cells on neuronal degeneration after hair cell loss. Journal of the Association for Research in Otolaryngology. 2005 Jun. 1; 6(2):136-47; Wang Y, Hirose K, Liberman M C. Dynamics of noise-induced cellular injury and repair in the mouse cochlea. Journal of the Association for Research in Otolaryngology. 2002 Sep. 1; 3(3):248-68).

### Example 1

### Graft Fabrication

A class of elastomers known as polyurethanes also have hydrogen-bonding “hard segments” that physically crosslink together the main polymer chain, also known as the “soft segments.” This pseudo-crosslinked structure gives polyurethanes their elastomeric properties that allow them to be flexed while still returning to their original conformation. In contrast to conventional elastomers, as mechanical forces are applied to polyurethanes, the hydrogen bonds are broken and more energetically favorable bonds are formed from the densification of the hard segments. This results in a polymer with hard segments densified perpendicular to the tensile direction and therefore polymer chains oriented parallel to the tensile direction. This new structure leads to a behavior known as “densification hardening” or “strain hardening” where the polymer becomes progressively stiffer as it begins to be stretched, as there begin to be stronger covalent bonds in tensile direction and weaker hydrogen bonds in the direction perpendicular to the applied force. This property of polyurethanes could potentially be extrapolated to filamentary extrusion printing—as the filaments are anchored to the substrate and the printhead moves, the filaments are essentially stretched in the direction in which the printhead is moving. As long as the final filament cross-sectional area is smaller than the nozzle diameter, there will be a degree of stretching occurring in the material.

Biodegradability of the material is crucial to enable the thickness of the graft to decrease as native tissue grows onto the surfaces of the graft. This property can enable a consistent thickness of the tympanic membrane to be maintained while the graft is remodeled. Thus, the soft segment can be designed to contain biodegradable bonds, such as PCL that contains ester bonds and nontoxic degradation byproducts. However, very few people have used biodegradable polyurethanes in 3D printing applications, and to date no one has used it specifically for the creation of anisotropic tissues. Most previously synthesized biodegradable polyurethanes are thermosets, meaning that as the temperature is increased, the material burns rather than melting. Thermoset polyurethanes have incredibly strong hydrogen bonds between hard segments, preventing polymer chains from reorganizing.

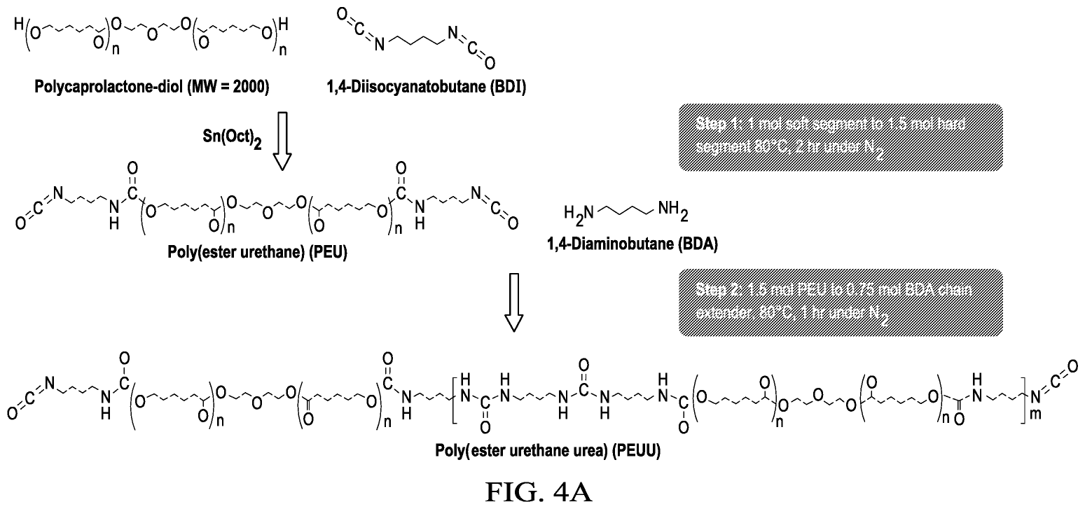

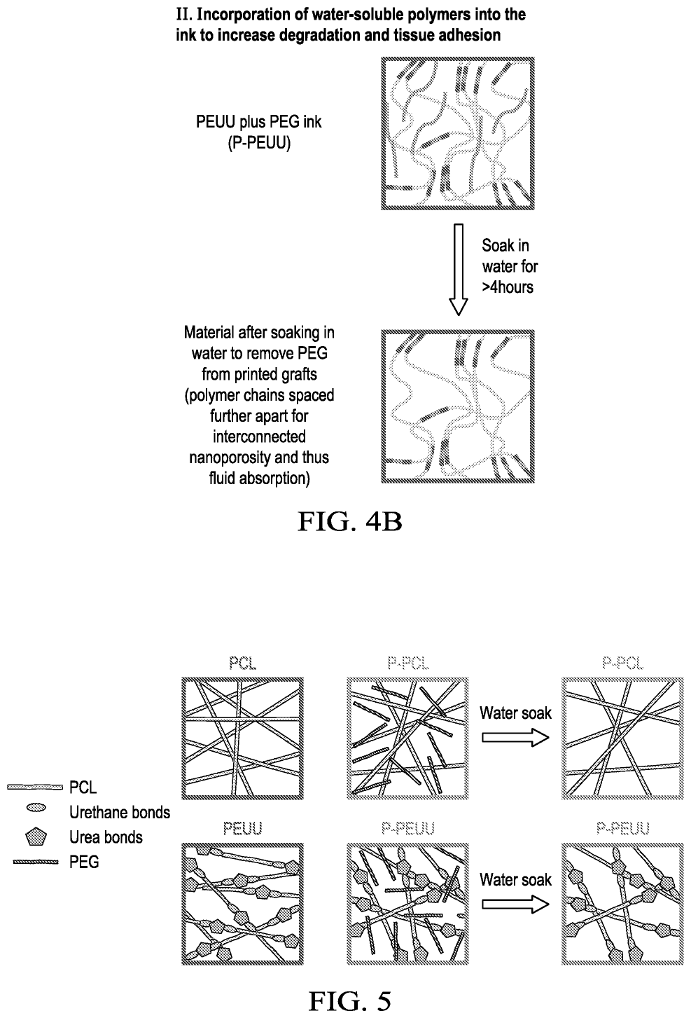

Thermoplastic polyurethanes that are able to melt at elevated temperatures have been designed for non-biomedical applications. To be able to liquify and extrude the polymer, the ratio of hard and soft segments must be modified to obtain a thermoplastic polyurethane with a reasonable melting temperature. This ratio is particularly challenging to optimize, as lowering the hard segment content has profound negative impacts on the mechanical properties of the polymer. Thus, a custom synthesis protocol was designed to create a poly(ester urethane urea) (PEUU) polymer that begins melting at around 40° C. with its viscosity decreasing with both increasing temperature and increasing shear strain, termed “shear thinning,” allowing it to be extruded through small nozzles while retaining robust mechanical properties at body temperature. Polyethylene glycol (PEG) was also mixed into the material prior to 3D printing to create a composite P-PEUU ink. Once the object was 3D-printed, the PEG was leached from the grafts by soaking them in water for at least 4 hours. This created an interconnected porous structure with high surface area for water and enzymes to degrade the grafts (FIGS. 4A-4B and 5).

To extrude the polymer, machinery was designed that is capable of both heating the material above its melting temperature and also applying pressure to the material, causing it to leave the printhead and form a filament. Thus, a small diameter steel nozzle was attached to a custom heated extrusion printhead that can heat the material to temperatures up to 120° C. The molten material has shear-thinning properties, thus as pneumatic pressure was applied to the top of the syringe containing the material, the viscosity lowers, and it flowed through the 200 μm inner diameter steel nozzle.

A combination of filament extension and high shear forces in the small inner diameter nozzle elongate the polymer chains along the print path. Tensile testing was performed on the material parallel and perpendicular to the print direction to determine the mechanical properties of the printed material. The elastic modulus of the P-PEUU was measured to be 70 megapascals along the print direction and 40 megapascals transverse to the print direction— within the 20 to 90 MPa range for the tympanic membrane. Additionally, it can stretch up to 150% of its original length without breaking, and it can be flexed onto itself and then easily return to its original conformation.

Finally, the programming of the final architecture for a tympanic membrane grafts was crucial to its function, particularly in replicating the circular and radial microarchitecture. The elastic modulus of the spiral and radial threads can widely differ, causing anisotropic mechanical properties. Thus, it allows the tympanic membrane to effectively capture sound.

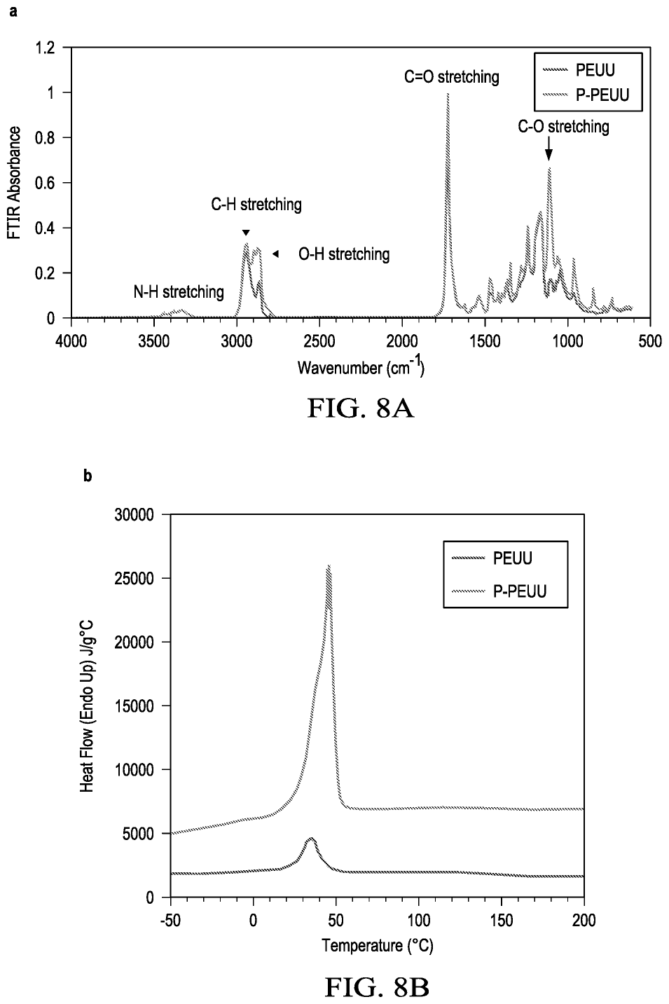

PEUU was successfully synthesized from PCL-diol soft segment, BDI hard segment, and BDA chain extender monomers in a 1:1.5:0.75 molar ratio to achieve a thermoplastic for use in HOT-DIW. As expected, PEUU contained urethane/urea bonds along its backbone with a characteristic C═O stretching peak observed at 1722 cm⁻¹ indicating the presence of ester and urethane bonds and another peak at 3330-3340 cm⁻¹ corresponding to N-H stretching in urethane and urea bonds (FIG. 8A; FTIR spectra of PEUU and P-PEUU, with peaks for relevant bonds indicated by arrows). Additionally, the FTIR spectrum of P-PEUU prior to PEG leaching indicates successful incorporation of PEG into this material, as reflected by an increased peak at 2500-3000 cm⁻¹ corresponding to O—H stretching and an increased peak at 1000-1200 cm⁻¹ corresponding to C—O stretching (arising from ether bonds along the PEG backbone) (Chieng, B. W., Azowa, I. N., Wan Md Zin, W. Y., & Hussein, M. Z. (2014). Effects of graphene nanopletelets on poly (lactic acid)/poly (ethylene glycol) polymer nanocomposites. In Advanced Materials Research (Vol. 1024, pp. 136-139). Trans Tech Publications Ltd). While PEUU has a melting temperature, Tₘ of 38° C., the presence of PEG leads to a secondary melting temperature peak, Tₘ, at 46° C. (FIG. 8B; heat flow during final heat ramp of a heat-cool-heat cycle between −50° C. and 200° C.; melting temperature, Tₘ, was defined as the maximal endothermic peak for each material).

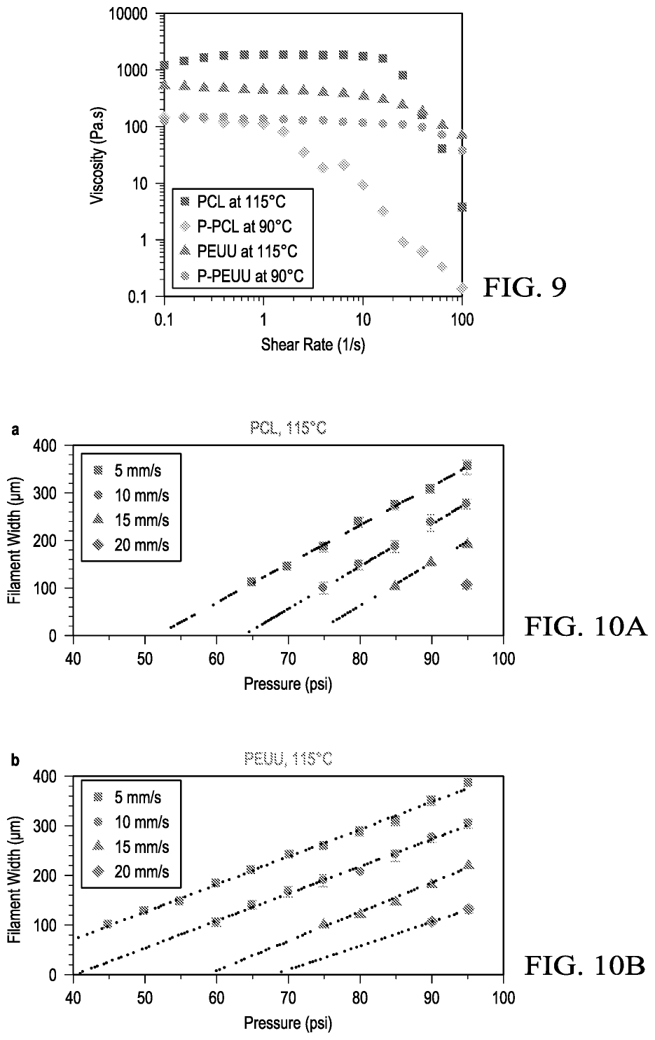

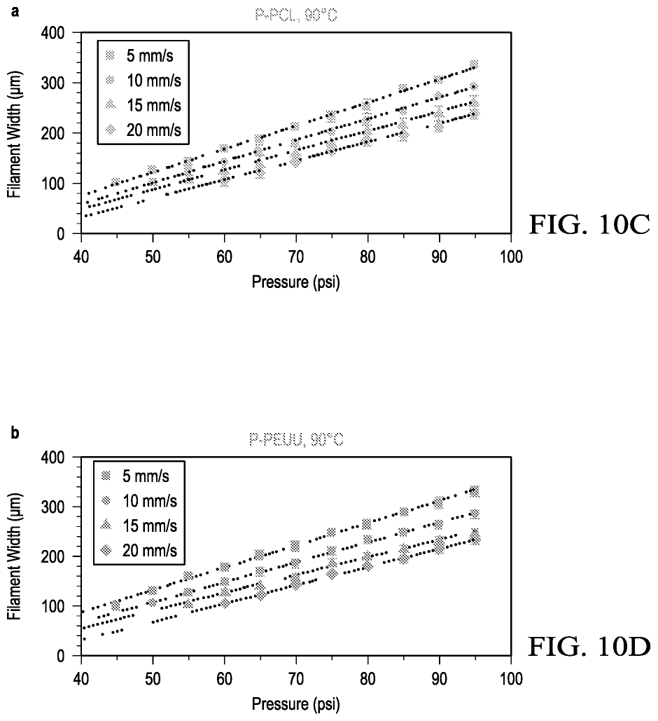

The apparent viscosity as a function of shear rate was reported for each ink used to produce biomimetic TM grafts via HOT-DIW (FIG. 9; inks tested include PCL, P-PCL, PEUU, and P-PEUU; note: P-PEUU and P-PCL inks contain 25 wt % PEG1500). These measurements are carried out at 90° C. for P-PCL and P-PEUU inks and 115° C. for PCL and PEUU inks, respectively, as this was deemed to be the optimal temperatures for patterning 100 μm filaments. While each of these inks exhibits the desired shear thinning behavior, the low shear viscosity varies from 100-1000 Pa·s, and the onset of shear thinning varies from roughly 1 s-1−20 s-1 depending upon the ink composition and temperature. Next, we explored the effects of printing speed and pressure on the width of the patterned filaments for inks extruded through a 200 μm nozzle at a layer height of 50 μm (FIGS. 10A-10B; (FIG. 10A) PCL inks extruded at 115° C., (FIG. 10B) PEUU inks extruded at 115° C., (FIG. 10C) P-PCL inks extruded at 90° C., and (FIG. 10D) P-PEUU inks extruded at 90° C.; measurements were taken from non-adjacent lines (n=6); error bars represent±SD). In all cases, a linear relationship between filament width and extrusion pressure, with increasing print speed and decreasing extrusion pressure resulting in filaments of smaller width. This information was used to identify print parameters that result in 100 μm wide filaments.

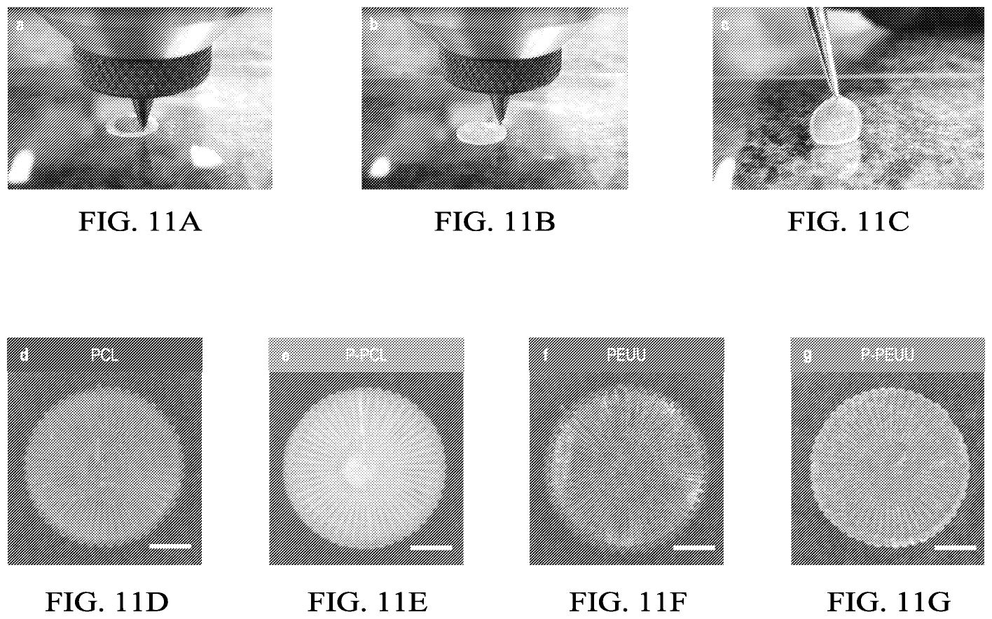

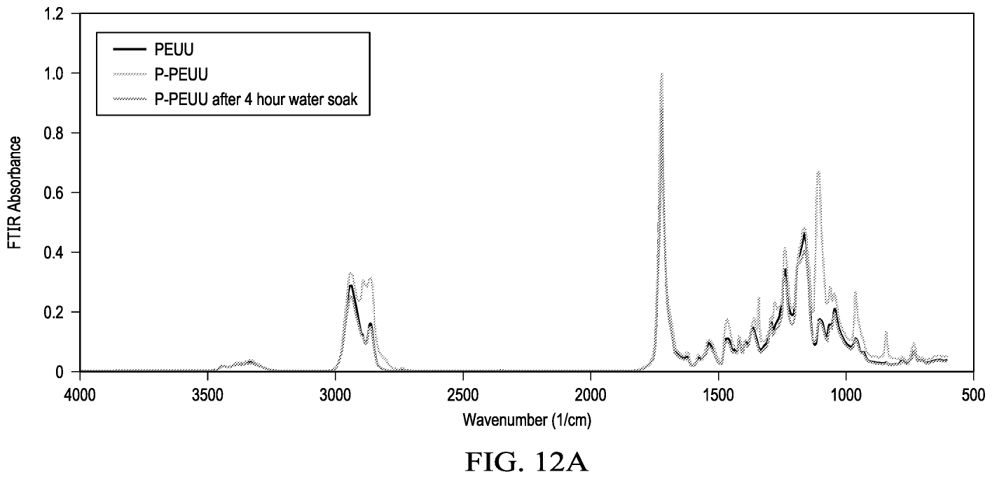

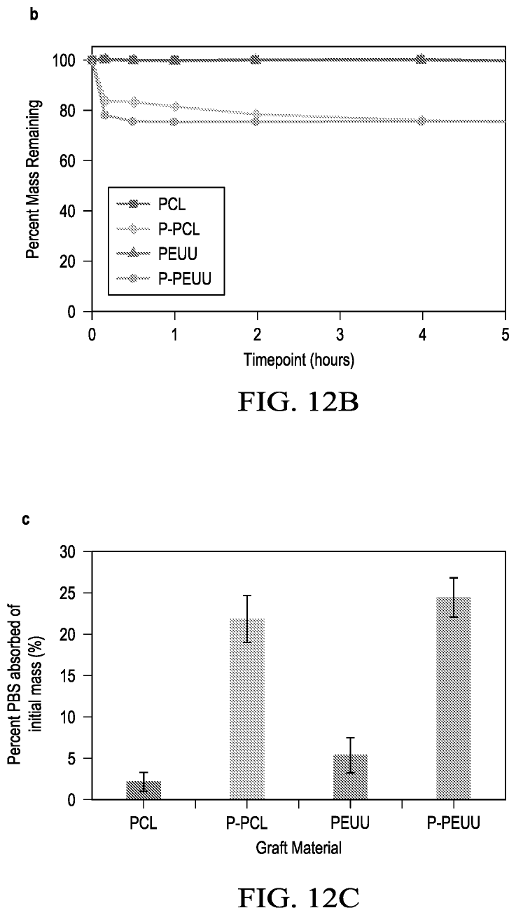

To demonstrate the suitability of each ink for HOT-DIW, we printed biomimetic TM grafts (50C/50R, 8 mm in diameter) from each ink (FIGS. 11A-11G; (FIG. 11A) 50 circular lines were 3D printed onto a glass substrate from the outer diameter inward; (FIG. 11B) 50 radial lines were 3D printed on top of the circular fibers from the center of the graft to the outer diameter; (FIG. 11C) due to rapid solidification following melt extrusion, grafts can be readily removed from the substrate following printing; (FIGS. 11D-11G) representative 50C/50R biomimetic TM grafts for the 4 biodegradable materials; all grafts were 3D printed utilizing a 200 μm inner diameter nozzle and a print speed of 20 mm/s; scale bars: 2 mm). After immersion in DI water for 4 h, the P-PCL and P-PEUU grafts lost ˜25% of their initial weight, which corresponds to their initial PEG content (FIG. 12B; change in mass of grafts after soaking in DI water at various timepoints (n=6); error bars represent±SD). As expected, pure PCL and PEUU grafts lost minimal weight over this time period, likely due to the removal of trace amounts of unreacted monomer or residual solvent. Full-thickness FTIR demonstrates that composition of P-PEUU grafts was nearly identical to the pure PEUU grafts after leaching process was complete, as noted by a concomitant decrease of peaks at 2500-3000 cm⁻ and 1000-1200 cm⁻¹ (FIG. 12A; FTIR spectra of PEUU grafts, P-PEUU grafts, and P-PEUU grafts that have been leached for 4 h in DI water; error bars represent±SD). The absorption of 20-25 wt % PBS in FIG. 12C (absorbance of PBS by grafts after PEG has been leached from the grafts (n=6); error bars represent±SD) was attributed to a nanoporous structure formed by PEG leaching in P-PCL and P-PEUU grafts.

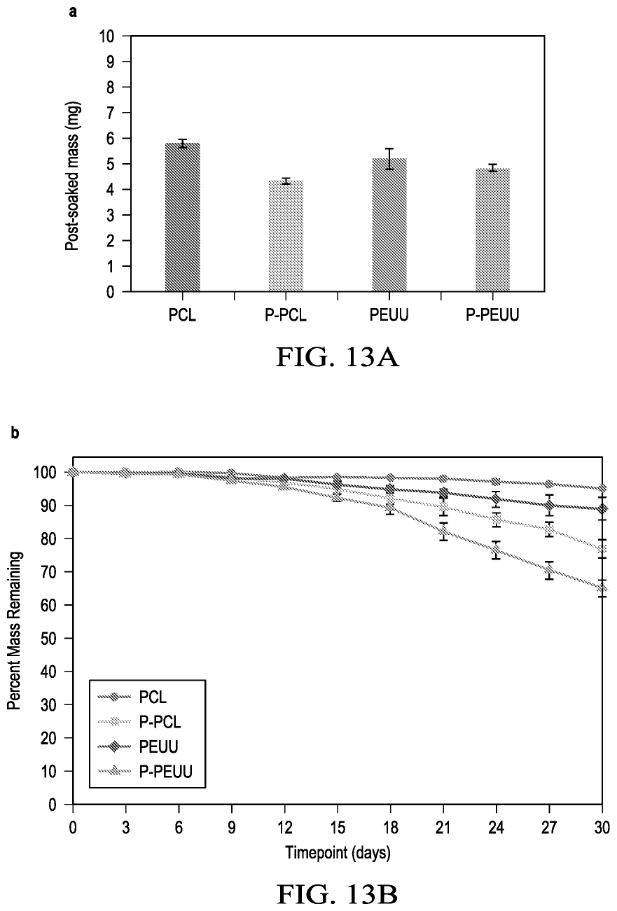

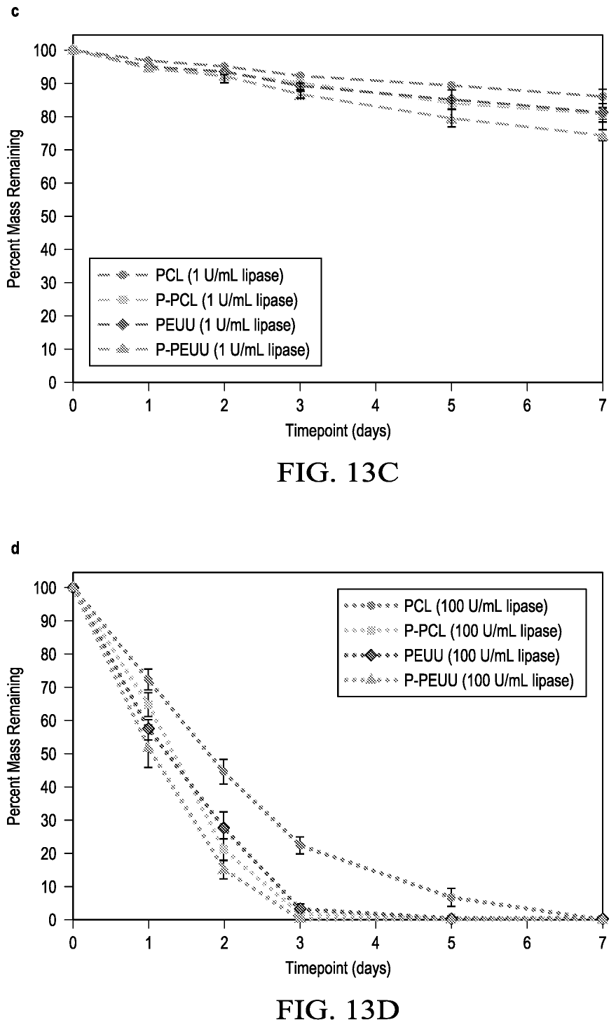

The biomimetic TM grafts (50C/50R) have a mass of 4-6 mg with the lower values associated with those subjected to PEG leaching (FIG. 13A). Graft degradation as a function of time in PBS, 1 U/mL lipase, and 100 U/mL lipase solutions were shown in FIGS. 13B-13D (all studies were conducted on a rocker at 60 rpm in an incubator at 37° C. with 5% CO₂ atmosphere), respectively. When immersed in PBS, the maximal degradation occurs for P-PEUU grafts, which loses ˜35 wt % of their initial mass after 30 days, followed by P-PCL grafts. The addition of lipase significantly enhances the degradation rate of all 4 grafts materials. For example, P-PEUU grafts lose ˜25 wt % of their initial mass after 7 days in 1 U/mL lipase. At even higher lipase concentrations, all grafts completely degrade after 7 days in 100 U/mL lipase. However, P-PEUU grafts exhibit the fastest degradation rates under all conditions. This enhanced degradation could be enabled by a nanoporous structure via both higher absorption of PBS and lipase solutions and also enhanced diffusion of degradation byproducts from the grafts.

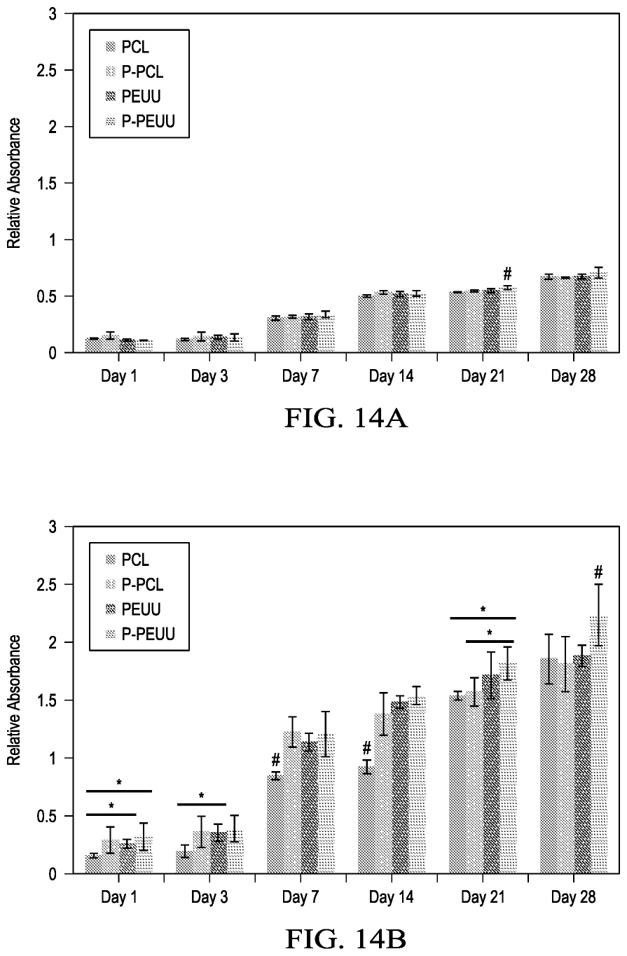

Both HEKs and GFP-HNDFs proliferate on biomimetic TM grafts (50C/50R) produced from all 4 inks (FIGS. 14A-14B; (FIG. 14A) HEKs, a human keratinocyte cell line, proliferate on all graft materials, as determine by an MTS assay; (FIG. 14B) GFP-HNDFs, a human fibroblast cell line, proliferate on all graft materials, as determine by an MTS assay; all samples were initially seeded with 100,000 cells on the top surface; error bars represent±SD; *p<0.05 from one specified group; #p<0.05 from all other groups in timepoint). There was no statistically significant difference between HEK proliferation on materials at most timepoints. At day 21, HEK proliferation was significantly higher (p<0.05) on the P-PEUU grafts than on grafts printed from the other 3 inks. At day 7, GFP-HNDF proliferation was significantly lower (p<0.05) on PCL grafts than P-PCL, PEUU, or P-PEUU grafts. At day 21, GFP-HNDF proliferation was significantly higher (p<0.05) than both PCL and P-PCL grafts. By day 28, GFP-HNDF proliferation was significantly higher (p<0.05) on the P-PEUU grafts than on grafts printed from the other 3 inks. Since PCL was widely used as a biocompatible polymer in medical implants, it was promising that grafts from novel P-PCL, PEUU, and P-PEUU inks promote cell proliferation at a similar rate. Additionally, as we observe P-PEUU grafts to have the highest proliferation at the most timepoints, this material was promising for in vivo applications.

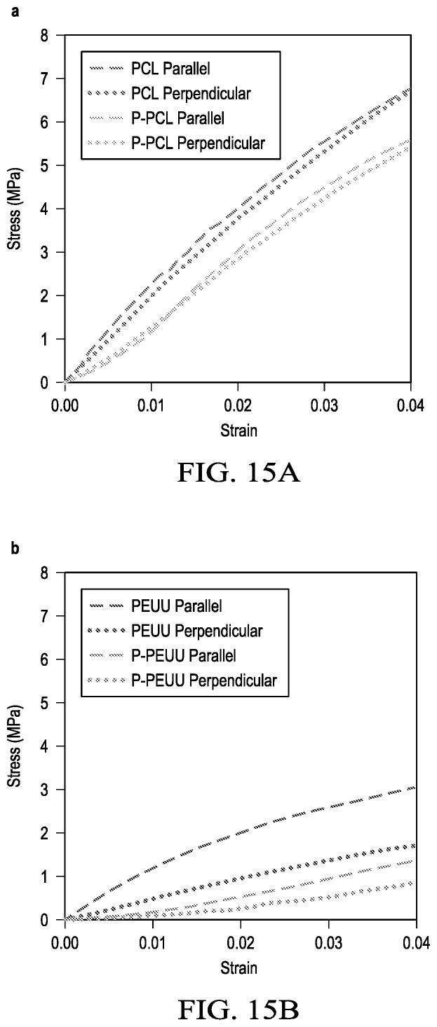

Tensile testing was successfully performed on printed PEUU, P-PEUU, PCL, and P-PCL dogbone samples soaked in PBS (prior to testing), in which the tensile load was applied in directions parallel and orthogonal to the print path (FIGS. 15A-15B; representative stress-strain curves for (FIG. 15A) PCL and P-PCL and (FIG. 15B) PEUU and P-PEUU for specimens printed parallel and orthogonal to the print path; comparison of average Young's Moduli of control and printed specimens; error bars represent±SD; *p<0.05; ***p<0.001). There was a statistically significant difference (p<0.001) between tensile specimens printed in a parallel versus an orthogonal orientation to the tension testing direction for both PEUU and P-PEUU. Specifically, the mean Young's modulus of PEUU specimens printed parallel to the testing direction was nearly 2× higher than the value measured orthogonal to this direction (E_{∥=}72.6±4.9 MPa, E_{⊥}=40.2±4.2 MPa), while the mean Young's modulus of P-PEUU specimens printed parallel to the testing direction was nearly 1.5× higher than the value measured orthogonal to this direction (E_{∥}=33.7±2.8 , E_{⊥}=23.8±1.5 MPa). PCL specimens also exhibited a statistically significant difference as a function of their orientation, but to a far lesser extent (p<0.05). The mean Young's modulus of PCL printed parallel to the testing direction was nearly 1.04× higher than the value measured orthogonal to this direction (E_{∥}=164.9±4.2 MPa, E₁₉₅ =158.3±2.9 MPa). There was not a statistically significant difference between P-PCL printed in parallel and orthogonal directions (E_{∥}=125.2±9.4 MPa, E_{⊥}=123.8±5.4 MPa). PEG removal from the P-PCL grafts reduces their Young's modulus values by 21.2% in the parallel direction and 24.1% in the orthogonal direction, whereas the mean Young's modulus of P-PEUU grafts decreased by 41.0% in the parallel direction and 53.6% in the orthogonal direction. Finally, the stiffnesses of the printed PEUU and P-PEUU grafts in both the parallel and orthogonal directions were in the range of that of the human TM, between 10-100 MPa. For comparison, the two control materials used in tympanoplasty, cadaveric temporalis fascia and Biodesign® porcine small intestinal submucosa (pre-soaked in PBS), have Young's moduli of Efascia=6.46±2.63 MPa and E_{BIODESIGN®}=20.6±3.82 MPa.

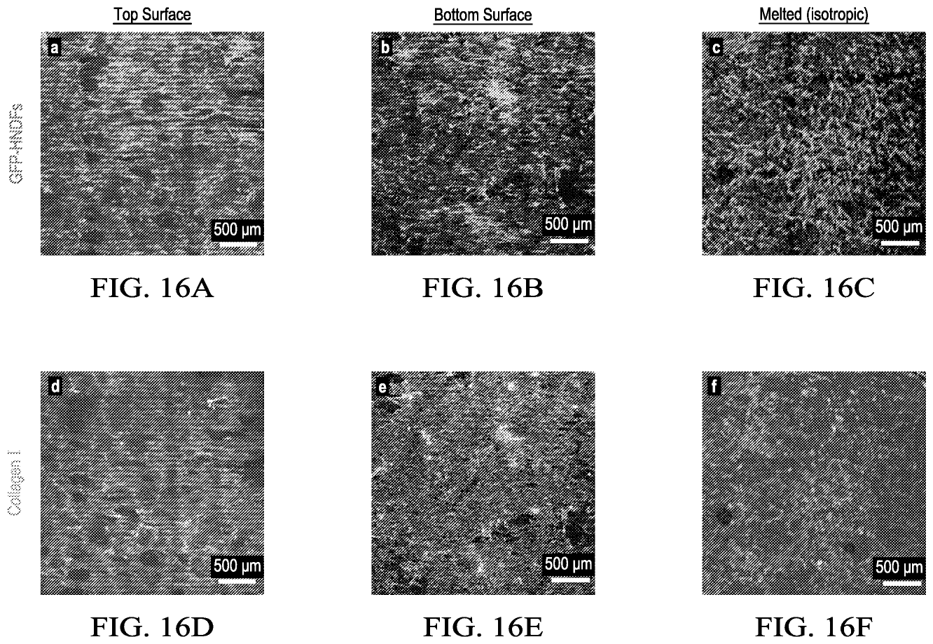

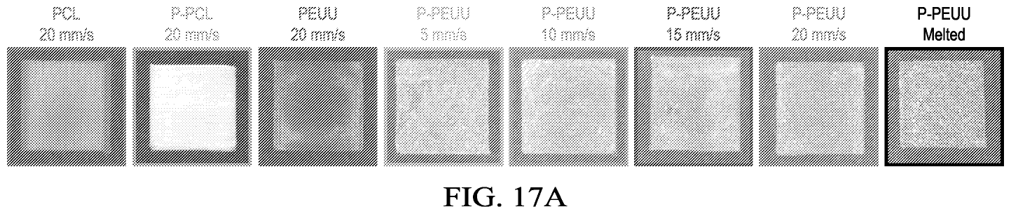

Next, we investigated the alignment of GFP-HNDFs and extracellular collagen I on biomimetic TM grafts printed from each ink via confocal imaging. GFP-HNDFs were seeded onto the top and bottom surface of grafts, as well as the bottom surface of melted grafts to “erase” anisotropy. Example confocal Z-stacks were shown wherein the channel indicating GFP-HNDF cellular alignment (FIGS. 16A-16C; confocal images) and the channel indicating extracellular collagen I alignment (FIGS. 16D-16F) on P-PEUU grafts printed at a speed of 20 mm/s to varying extents depending upon the top surface of the grafts, bottom (topographically more flat) surface of the grafts, or the bottom surface of grafts that have been melted to “erase” any graft anisotropy (note: the center 6×6 mm region of each graft whose total size was 8×8 mm was used in ImageJ Directionality analysis, but only a 3×3 mm region was shown in this figure to more clearly demonstrate cell morphology in the images).

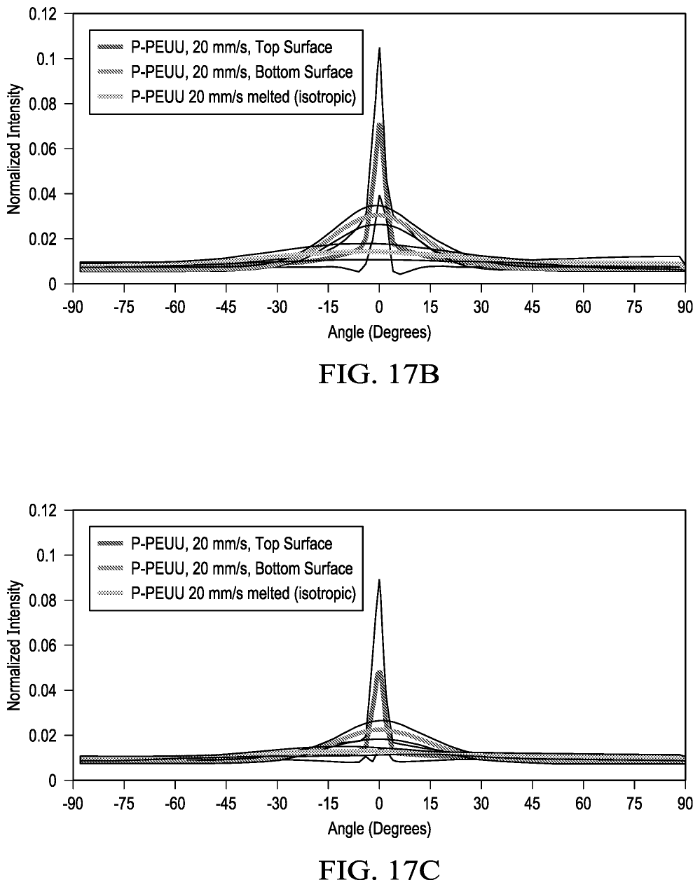

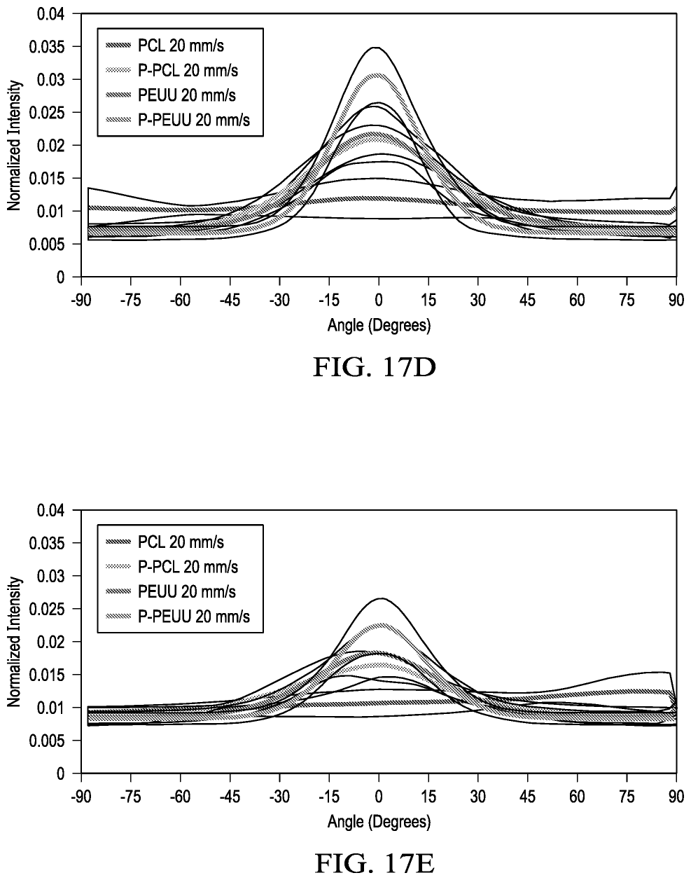

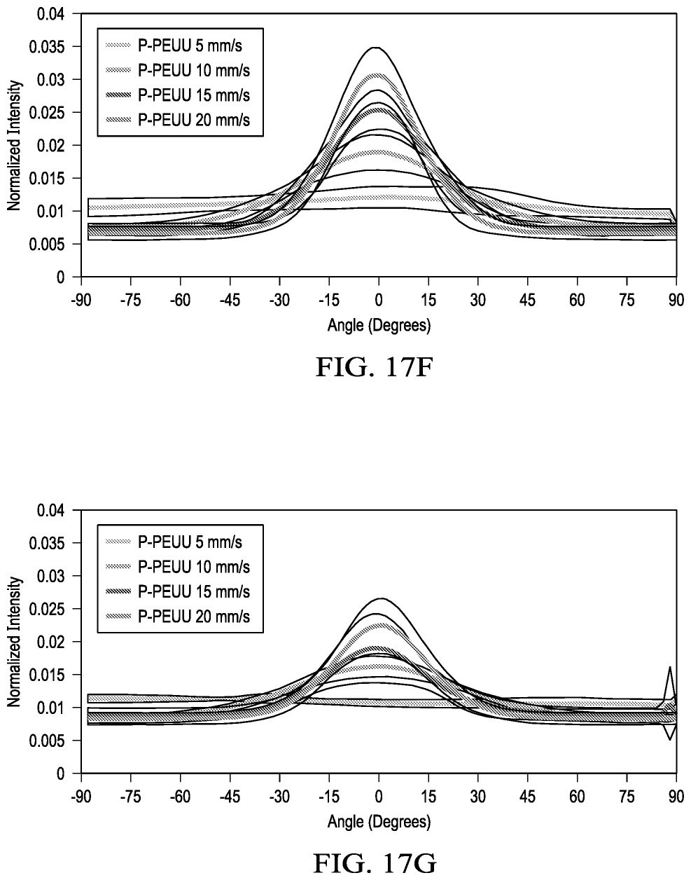

GFP-HNDFs were successfully seeded onto a variety of 8×8 mm square grafts printed from 200 μminner diameter nozzles while maintaining a layer height of 50 μm and a filament width of 100 μm(FIG. 17A; optical microscopy images of 8×8 mm square grafts 3D printed from various materials and print speeds while maintaining a print height of 50 μmand filament width of 100 μm), as described previously. An assessment of these two channels separately with ImageJ Directionality analysis quantifies this cellular and extracellular matrix protein alignment, whereby the print path corresponds to a direction of 0°. The relative impacts of topography, material, and print speed on the cellular and extracellular collagen I alignment were quantified and compared between graft groups (n=6 each).

The highest extent of GFP-HNDF and collagen I alignment was observed on the top surface of P-PEUU grafts printed at a speed of 20 mm/s, as compared to those seeded on the bottom (topographically flatter) surface of the grafts or the bottom surface of grafts that have been melted to “erase” any graft anisotropy. Alignment was highest for cells seeded on the top surface of these grafts, whereby filaments produce distinct topographical features, reaching an average normalized intensity of 0.0841 at its peak for the GFP-HNDF channel (FIG. 17B; GFP-HNDFs) and 0.0556 at its peak for the collagen I channel (FIG. 17C; Collagen I). However, even on the bottom (topographically flatter) surface of the grafts, there was distinct GFP-HNDF and collagen I alignment. This directional alignment disappears for cells seeded on the bottom surface of grafts that have been melted to “erase” any anisotropy imparted into the grafts by the printing process.

To decouple the impact of macroscale filament topography on cellular and extracellular matrix protein alignment on the grafts, comparisons were made between grafts of different materials whereby cells were seeded only onto the bottom (topographically flatter) surface of grafts. When comparing the 4 biodegradable inks (PCL, P-PCL, PEUU, and P-PEUU) printed at a speed of 20 mm/s, we observed the highest extent of both GFP-HNDFs and collagen I alignment for fibroblasts seeded onto P-PEUU grafts, reaching a normalized intensity of 0.0306 at its peak for the GFP-HNDF channel (FIG. 17D; GFP-HNDFs) and 0.0222 at its peak for the collagen I channel (FIG. 17E; Collagen I). For PCL grafts printed at a speed of 20 mm/s, there was a very low extent of alignment in the GFP-HNDF channel observed, reaching a normalized intensity of 0.0118 at its peak, and almost no obvious direction of alignment observed in the collagen I channel.

For a similar comparison of the impact of print speed, P-PEUU grafts were successfully printed via HOT-DIW with varying print speeds, using the pneumatic expression pressures determined previously in FIGS. 10A-10B: 5 mm/s at 45 psi, 10 mm/s at 50 psi, 15 mm/s at 55 psi, and 20 mm/s at 60 psi. We observe that increasing the print speed of P-PEUU during HOT-DIW while maintaining the filament diameter by increasing extrusion pressure increases alignment of both the cells themselves via GFP-HNDF channel (FIG. 17F; GFP-HNDFs) and the collagen deposition via the collagen I channel (FIG. 17G; Collagen I). Overall, alignment of collagen I was lower than intracellular GFP alignment itself for all samples. Note, in 17A-17G, angles of −90° and 90° correspond to the direction orthogonal to the print path, while an angle of 0° corresponds to the direction parallel to the print path. Narrower and taller distribution around 0 corresponds to a highest extent of alignment in the direction the print path.

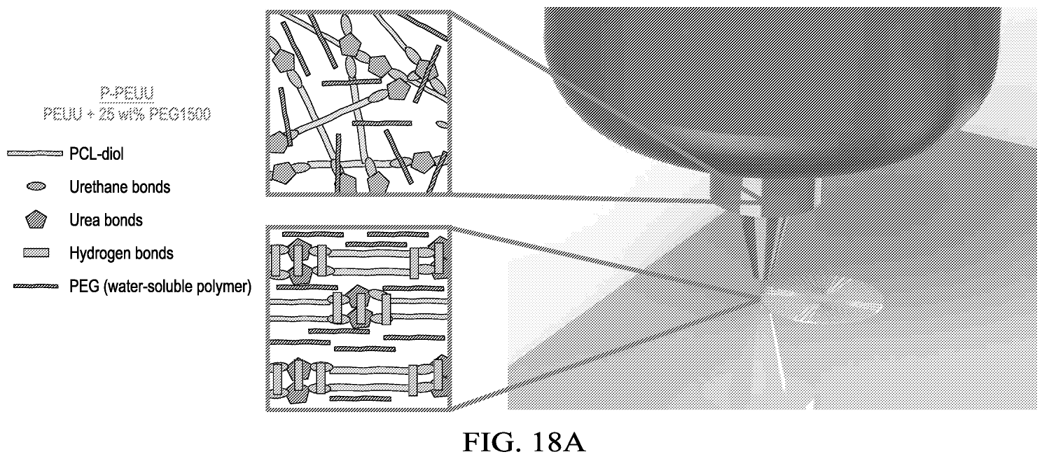

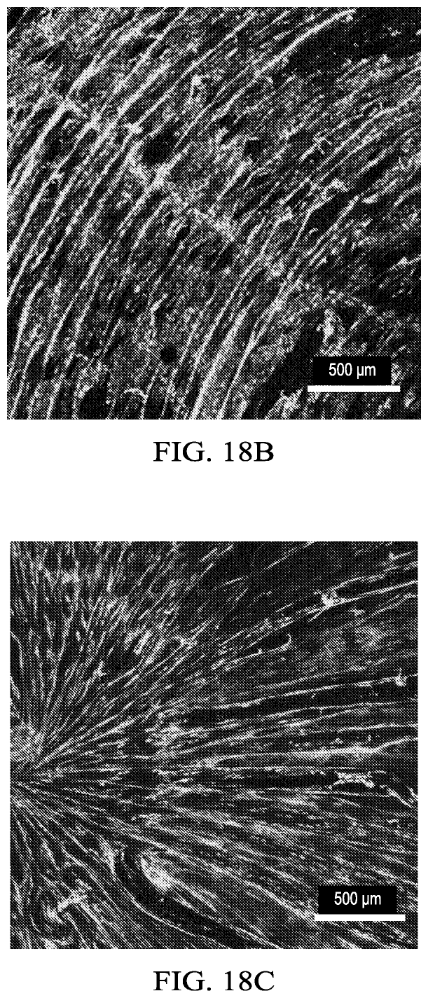

Extrusion processes through small diameter nozzles, such as HOT-DIW, cause high shear stress in the nozzle. This shear stress increases with increasing print speeds while maintaining filament diameter. This shear stress could potentially cause molecular alignment of the polymer along the print direction, which could then be maintained by physical bonding between hard segments (FIG. 18A; expected printing behavior of inks upon extrusion, whereby hydrogen bonding occurs between urethane and urea bonds;

fugitive PEG was shown in the enlarged boxes). We observed GFP-HNDF and extracellular collagen I alignment along the print path of biomimetic P-PEUU 50C/50R grafts printed at 20 mm/s, after 7 days of GFP-HDNF growth, on both the bottom, circular surface (FIG. 18B) and the top, radial surface (FIG. 18C). As the topography of the bottom surface of the grafts was relatively flat, cellular alignment in the P-PEUU inks may be attributed to the anisotropic properties of the grafts.

### Example 2

### In Vitro and In Vivo Testing of 3D Printed Graft

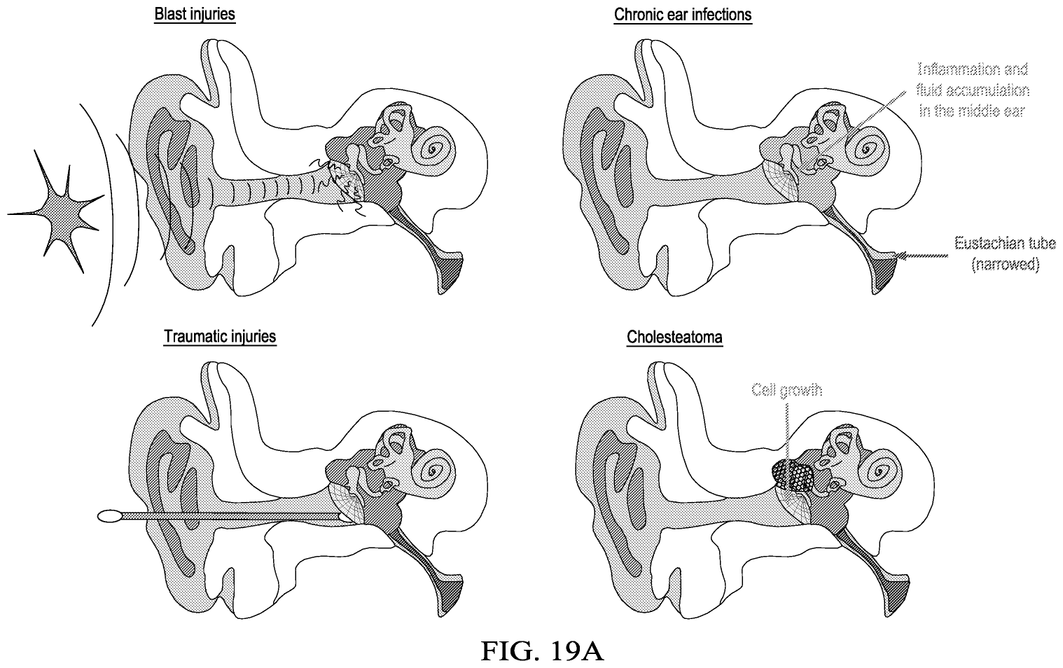

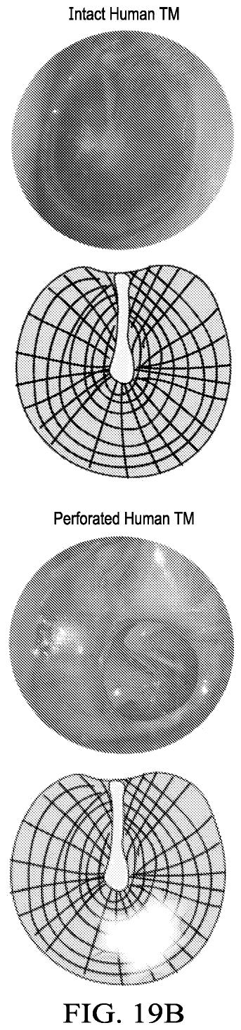

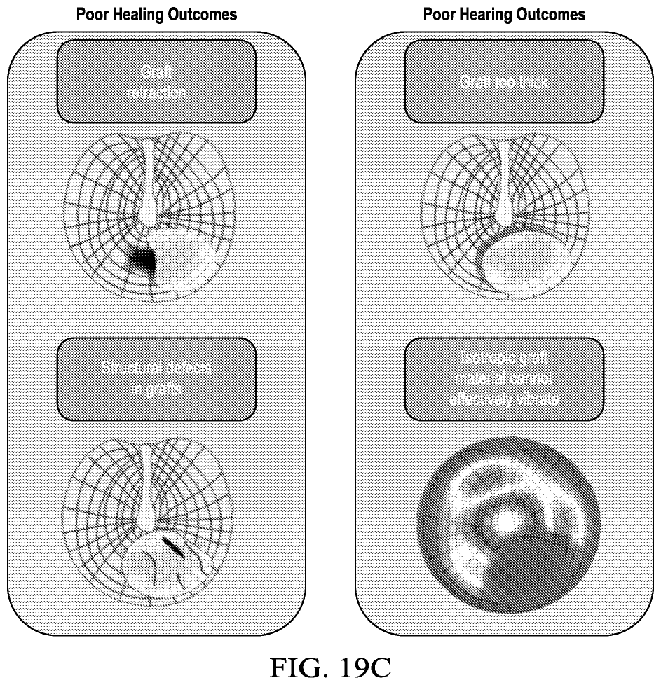

As noted above, hearing loss is a prevalent problem, especially in servicemen/servicewomen. FIG. 19 shows what happens to ears after suffering a blast injury (see FIG. 19A) and specifically tympanic membranes (FIG. 19B). If TMs are not properly healed, poor hearing outcomes result (FIG. 19C). Given the ideal resultant features of the inks described herein, the potential for using the inks described herein for blast victims were tested both in vivo and in vitro.

To create grafts with a circular and radial fibrous microarchitecture, the Aerobasic G-code programming language allowed for precise geometries to be patterned and their dimensions to be tuned. Thus, the overall diameter, thickness, number of circular lines, and number of radial lines were rapidly altered, allowing for testing various design parameters and potential customization of the grafts. Circular lines were printed first with each anchored to the substrate. Then, radial lines were patterned on top with equal spacing between (see, e.g., FIG. 11).

Approval for animal surgery is obtained and performed in accordance with the guidelines of the Institutional Animal Care and Use Committee (IACUC) at Massachusetts Eye & Ear. Chinchillas (Chinchilla lanigera, ˜500 grams each, females) were anesthetized and monitored. Animals undergo baseline ABR and DPOAE testing in a sound treated booth as described in the below section. For control materials, fascia grafts were harvested from the superficial surface of the masticator muscle on chinchillas with a radius of approximately 8 mm. Biodesign® grafts were cut with a radius of approximately 8 mm as a second control material. Following hearing testing, the TM is visualized via using a rigid 0° and 30° Storz Hopkins® rod endoscope with a light source and camera (KARL STORZ, Germany). A low-temperature thermal myringotomy loop (Bovie Medical, USA) is used to create a 50% perforation on the inferior portion of the pars tensa (Amoils C P, Jackler R K, Milczuk H, Kelly K E, Cao K. An animal model of chronic tympanic membrane perforation. Otolaryngology—Head and Neck Surgery. 1992 January; 106(1):47-55). The inner mucosal layer of the TM is removed using a 1 mm hook, and radial orientated incisions were made in the remnant TM. This process allows for infolding of epithelialized flaps. An identical procedure is then repeated on the contralateral ear. Weekly endo-otoscopy (weeks 1-4) is performed to ensure perforations remain stable and free of infection (FIGS. 20A-20E).

The animals undergo transcanal endoscopic tympanoplasty utilizing biomimetic P-PEUU 50C/50R TM grafts in one ear (n=20) with control autologous fascia grafts (n=8) or Biodesign® grafts (n=12) in the contralateral ear (FIGS. 20F-20H). Grafts were placed in an underlay, transbullar fashion. Following graft placement, the middle ear is packed with absorbable Gelfoam® (Pfizer, USA) for graft stabilization during healing. Antibiotic ofloxacin drops were applied to both ears daily starting 1 week following the procedure. All treated and control ears undergo regular otoscopic evaluation to determine healing rates. After 3 months, a binary decision is made as to whether the tympanoplasty is successful in closing the perforation based on endoscopic examination.

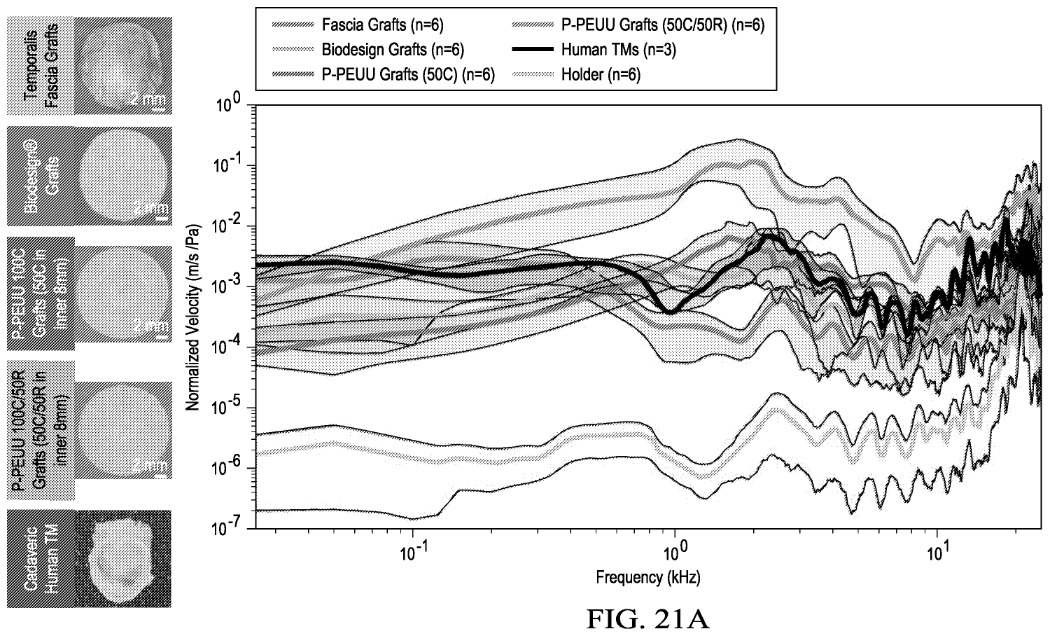

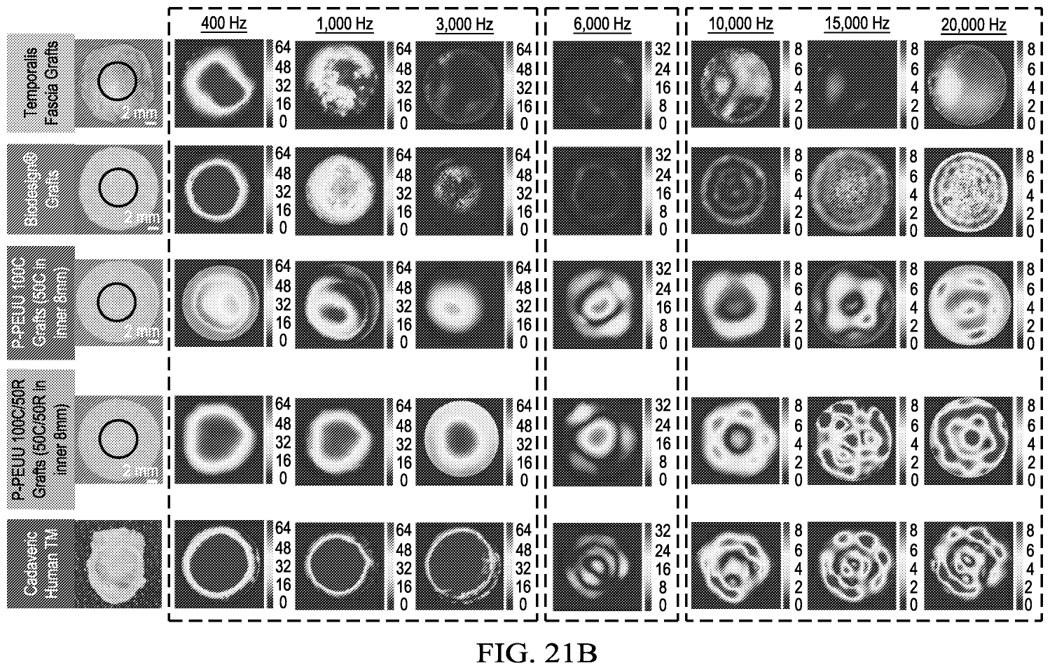

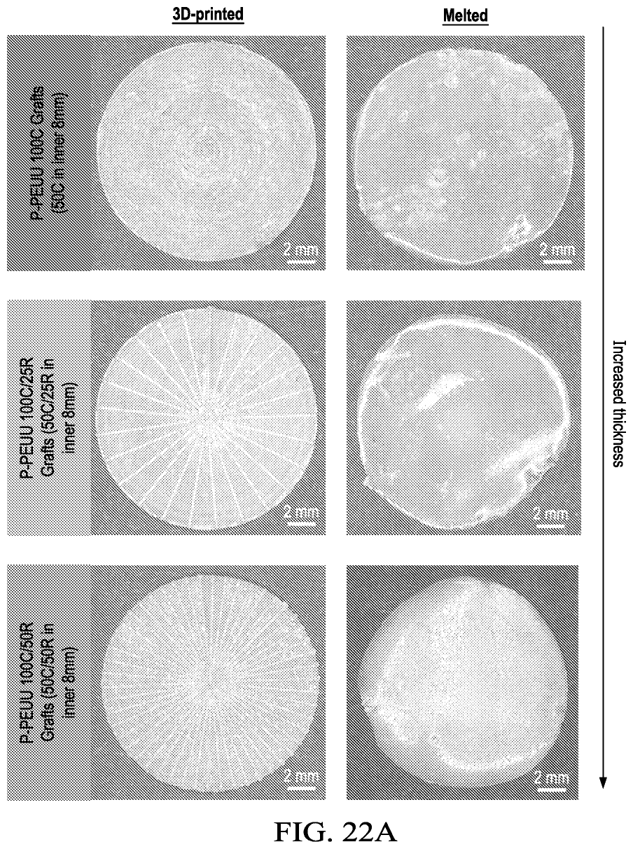

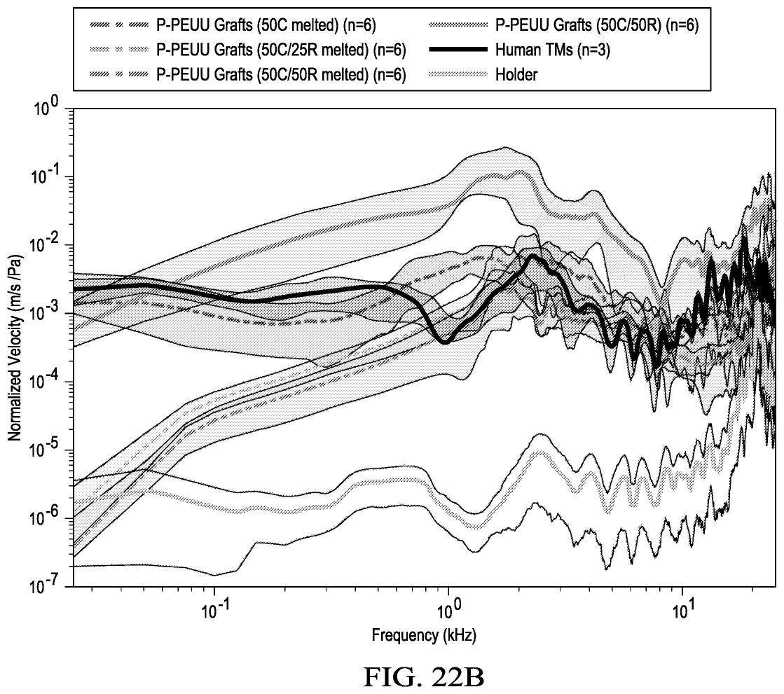

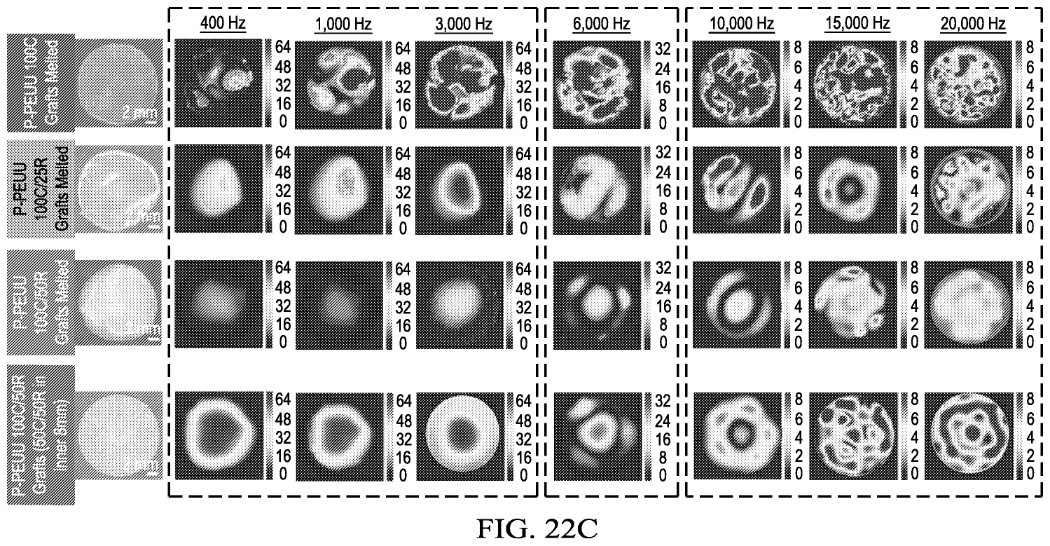

The acoustic implications for this circular and radial architecture were studied in vitro by mounting them circumferentially onto a custom holder and playing sound behind them. The first test, laser Doppler vibrometry (LDV), quantified sound-induced motion over a broad frequency range at the graft's center. The second test, digital opto-electronic holography (DOEH), resolved full-field motion patterns at discrete frequencies. Control materials of fascia and small intestinal submucosa (SIS) tissue as well as 3D-printed 50C (50 concentric circles) and 50C/50R (50 concentric circles and 50 radial lines) P-PEUU TympanInk grafts were mounted and tested. The resultant LDV vibration velocities normalized by sound pressure show that the 3D-printed 50C/50R grafts show improved motion over both control materials as well as the50C grafts, demonstrating the importance of radial stiffness for allowing sound conduction at both low and high frequencies (FIGS. 21A and 21B). Additionally, DOEH motion patterns demonstrate complex modes of motion at high frequencies for the 50C/50R grafts, unlike the control grafts. To control for any changes in thickness imparted by a radial layer, grafts from the same print batch were melted, re-solidfied, and the PEG leached to create isotropic sheets of P-PEUU material in the same thickness as the 3D-printed grafts. LDV demonstrates that the sound-induced velocity of the 50C/50R grafts significantly drop after this architecture is removed (FIGS. 22A-22C). Among the isotropic melted grafts, thinner grafts printed with less material (50C), have a higher velocity than the thicker, melted and isotropic 50C/25R or 50C/50R grafts. However, even the thin melted 50C grafts did not have as high of a velocity as the 3D-printed 50C/50R grafts, once again demonstrating the importance of this radial stiffness for efficient sound conduction.

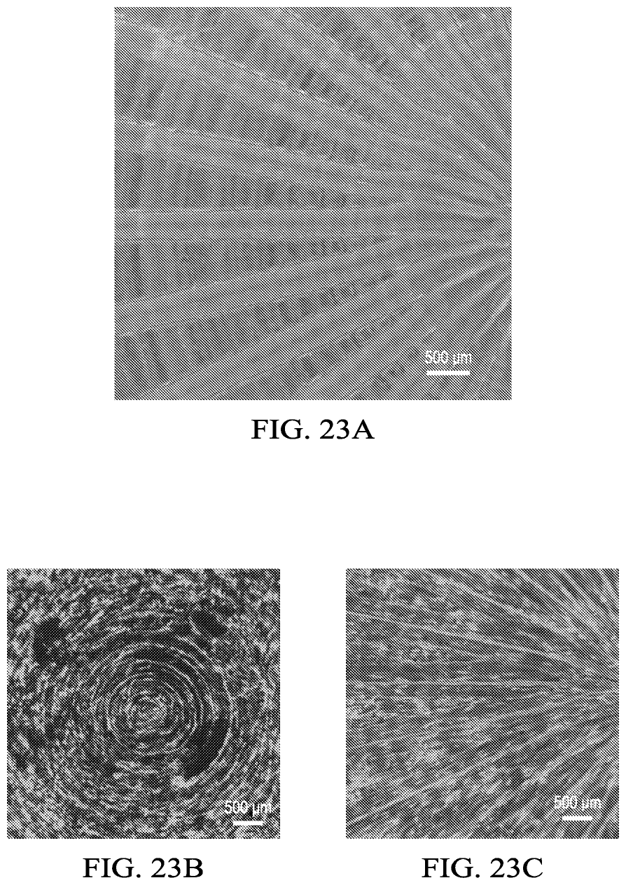

Because of the importance of this radial stiffness in the tympanic membrane, it is crucial for this architecture to remain even after the grafts degrade and were replaced by native tissue. To control this alignment via mechanotransduction, the ideal tympanic membrane graft will be stiffer along the circular and radial directions. This should cause cells to elongate along the complex printed directions and therefore lay down collagen in an architecture that resembles the original print path. The elastic modulus of the 3D-printed P-PEUU ink was almost double parallel to the print path than perpendicular to the print path, thus, one would expect cells spreading to occur in the direction parallel to the printed filaments. Indeed, one does observe alignment of human fibroblasts under in vitro cell culture conditions, even on the topographically flat surface of the grafts (FIGS. 23A-23C). The fibroblasts also laid down collagen along the direction of the print path. This can be extended to complex geometries as well, including circular and radial patterns, showing promise for anisotropic in vivo tissue remodeling of circularly and radially printed tympanic membrane grafts.

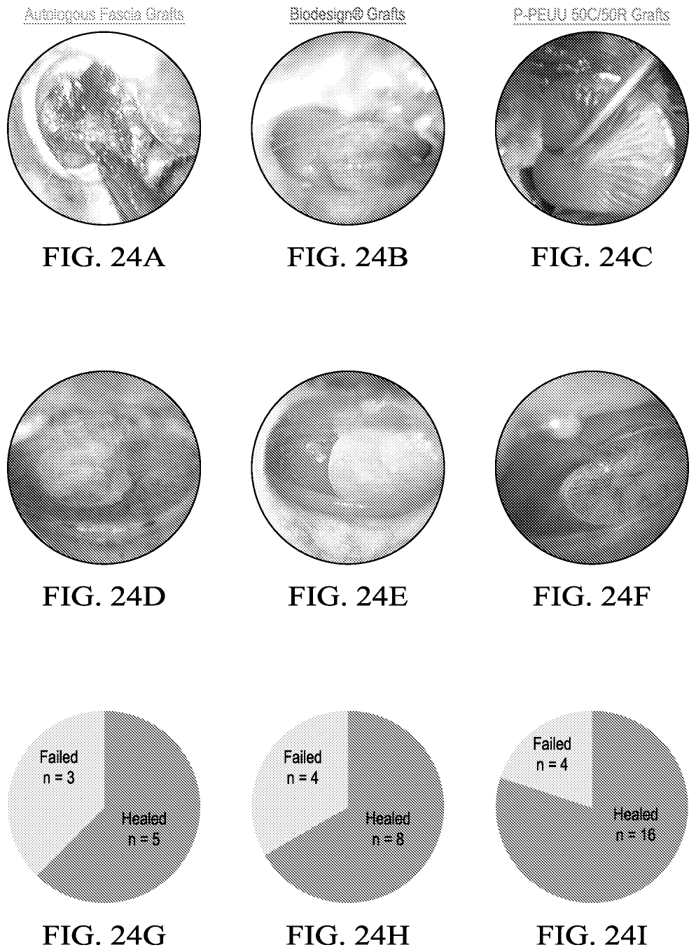

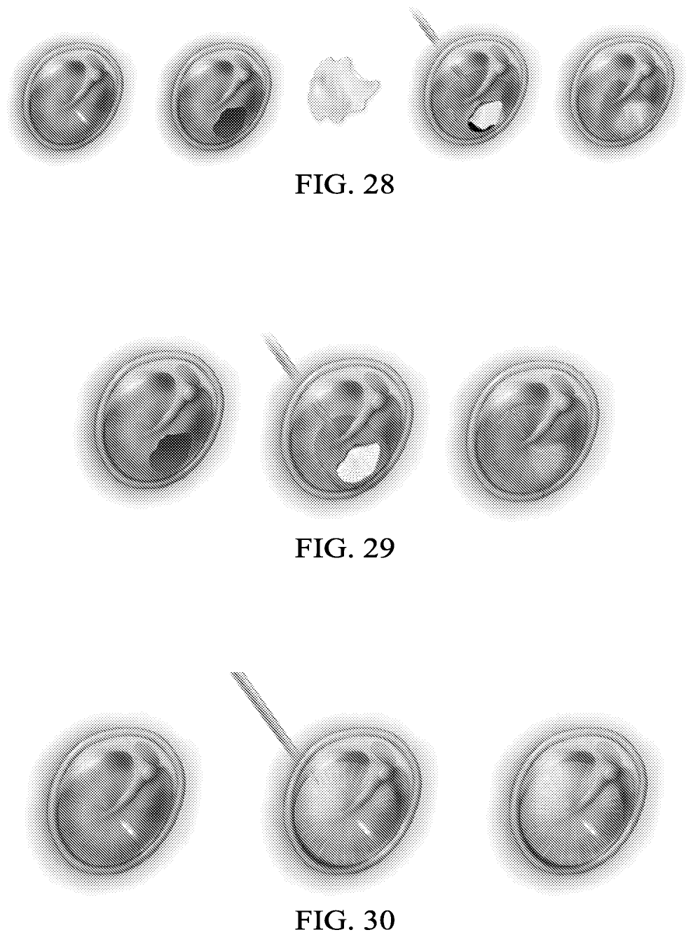

Given the promising in vitro acoustic results for biomimetic P-PEUU 50C/50R grafts, we focused next on their healing and hearing outcomes in in vivo studies using chinchilla models for chronic TM perforations. Chinchillas with chronically perforated TMs successfully underwent transbullar underlay tympanoplasty procedures with biomimetic P-PEUU 50C/50R grafts and two controls: autologous temporalis fascia grafts and Biodesign® grafts. Notably, both the P-PEUU 50C/50R and Biodesign® grafts exhibit good mechanical properties and handling compared to temporalis fascia grafts, which must be completely desiccated to appropriately position the graft adjacent to the remnant TM (FIGS. 24A-24C). Next, we explored the integration of the biomimetic P-PEUU 50C/50R, autologous temporalis fascia and Biodesign® grafts using serial otoendoscopy. Representative otoscopic images of healed grafts (after 3 months) show their final TM structure (FIGS. 24D-24F). The number of failed grafts and healed grafts for (FIG. 24G) temporalis fascia grafts, (FIG. 24H) Biodesign® grafts, and (FIG. 241) 3D-printed P-PEUU 50C/50R grafts). While fascia and Biodesign® grafts were well integrated, the original grafts were still readily identified without any indication of degradation or remodeling. By contrast, native tissue ingrowth is observed within the circular and radial architecture of the biomimetic P-PEUU 50C/50R grafts. This promising result indicates that native cells can remodel the biodegradable material into native tissue. Moreover, ingrowth of native vasculature via angiogenesis is observed, consistent with the vascular ingrowth seen in biodegradable polyurethanes. The overall healing rate differs between graft types, with the biomimetic P-PEUU 50C/50R grafts (n=20) exhibiting the highest rate of successful tympanoplasty, at 75% of implanted grafts (FIGS. 24G-24I). By comparison, commonly used fascia (n=8) and Biodesign® (n=12) grafts for tympanoplasty had lower rates of perforation closure, at only 62.5% and 66.6%, respectively. Graft failures arose due to graft retraction, re-perforation, and infection of the chinchilla TM post-surgery. Thus, unlike autologous tissue grafts which do not degrade or remodel into tympanic membrane tissue (FIG. 28), biomimetic grafts made with the invention present the possibility to reconstruct the tympanic membrane in a biomimetic fashion (FIG. 29).

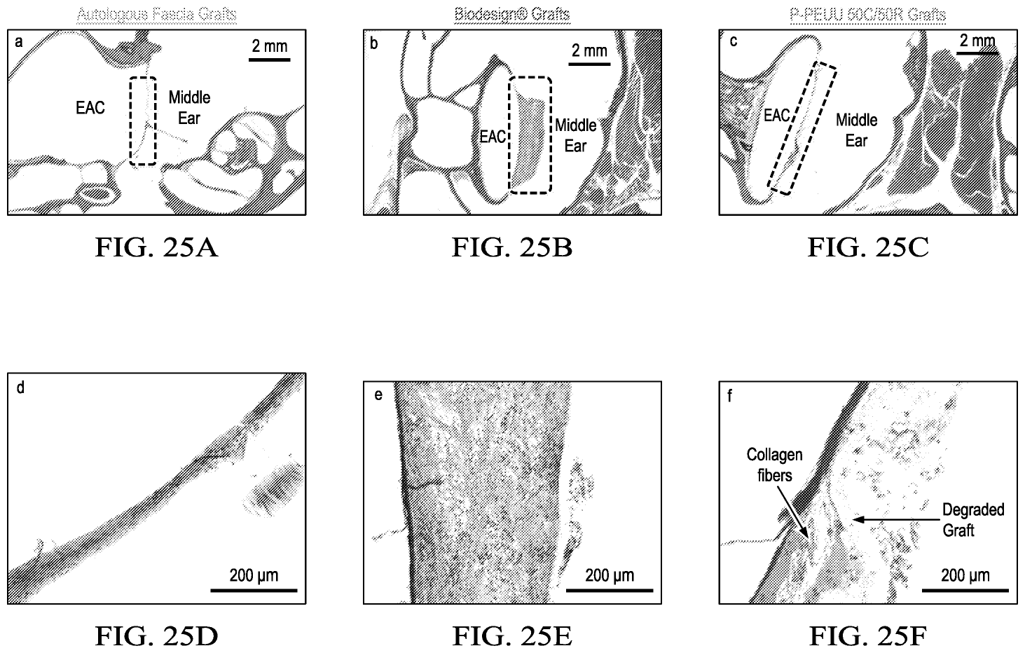

Stained histological sections of representative biomimetic P-PEUU 50C/50R, fascia, and Biodesign® grafts were shown in FIGS. 25A-25F. While all sections show perforation closure and restoration of the boundary between the external auditory canal (EAC) and the middle ear space, the cross-sections of the remodeled TMs significantly differ. As native cells grow into the grafts to close the TM perforation, lack of graft material degradation leads to an increased thickness and thus lower sound-induced motion, particularly at low frequencies. Although it is difficult to make quantitative thickness measurements due to potential shearing of the grafts during histological slicing and absorbance of fixation solutions, their overall structure can be compared. While fascia grafts maintain a thin structure with a keratinocyzed epidermal layer adjacent to the EAC, there were no signs of remodeling into the lamina propria, with grafts retaining their original linear structure. Similarly, Biodesign® grafts show a keratinocyzed epidermal layer adjacent to the EAC; however, the thickness of these grafts is substantially greater than that of the native TM, at around 200-300 μm. In contrast, P-PEUU 50C/50R grafts show native cellular ingrowth on both the medial and lateral sides of the graft, with arranged collagen fibers being deposited. The partially degraded P-PEUU material has a measured thickness of 32 μm±11 μm (taken across 6 sections). Given their original thickness of 107±4 μm, this represents a 70% reduction after 3 months of implantation. We expect this thickness to reduce further over time as the P-PEUU material fully degrades.

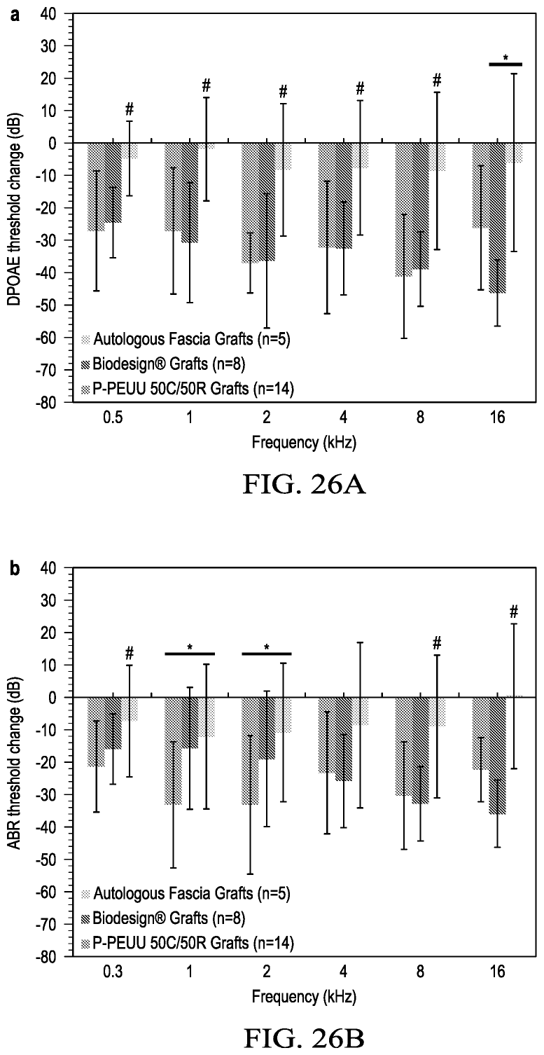

To complement the healing outcomes of biomimetic P-PEUU 50C/50R, fascia, and Biodesign® grafts, we also evaluated the hearing outcomes of these TM graft materials using DPOAE and ABR. Threshold values at 3 months following tympanoplasty were subtracted from initial hearing values with an intact TM (FIGS. 26A-26B). Since higher threshold values indicate higher sound pressure levels required for electrophysiological feedback indicating hearing, values closer to 0 indicate restoration of hearing close to baseline levels with an intact TM, whereas more negative values represent greater hearing loss following tympanoplasty. There is a large variation between animals due to anatomical differences and complexity of the surgical procedure. Thus, statistical comparisons were performed.

In chinchillas whose TMs were repaired with P-PEUU 50C/50R grafts, DPOAE hearing thresholds were restored closer to normal in a statistically significant (p<0.05) amount compared to both fascia and Biodesign® grafts at all frequencies tested besides 16,000 Hz, whereby hearing threshold restoration is significantly better than fascia grafts but not significantly better than Biodesign® grafts, despite being better on average. Analysis of ABR results showed similar improvement in hearing restoration for biomimetic P-PEUU 50C/50R grafts. In chinchillas whose TMs were repaired with those grafts, hearing is restored at a statistically significant and improved level (p<0.05) compared with fascia grafts at lower frequencies of 1000 and 2000 Hz. Additionally, there is statistically significantly (p<0.05) restored hearing in chinchillas whose TMs were repaired with P-PEUU 50C/50R grafts compared with outcomes for both fascia grafts and Biodesign® grafts at higher frequencies of 8,000 and 16,000 Hz. Interestingly, the average ABR thresholds at 3 months following tympanoplasty procedures for 16,000 Hz were better than those with the chinchilla's normal, intact TM prior to perforation, indicating an overall improvement (beyond baseline) of hearing function at high frequencies.

Control grafts exhibited no statistically significant difference (p<0.05) between hearing outcomes for fascia and Biodesign® grafts in either ABR or DPOAE analysis, although as a general trend, average ABR hearing thresholds were restored closer to normal at lower frequencies (400, 1000, and 2000 Hz) for chinchillas undergoing tympanoplasty with Biodesign® grafts. In contrast, average ABR and DPOAE hearing thresholds were restored closer to normal at higher frequencies (4000, 8000, and 16,000 Hz) for chinchillas undergoing tympanoplasty with fascia grafts as compared to those with Biodesign® grafts. This may be due to the thinner remodeled TM with an overall lower mass, as seen in FIG. 25.

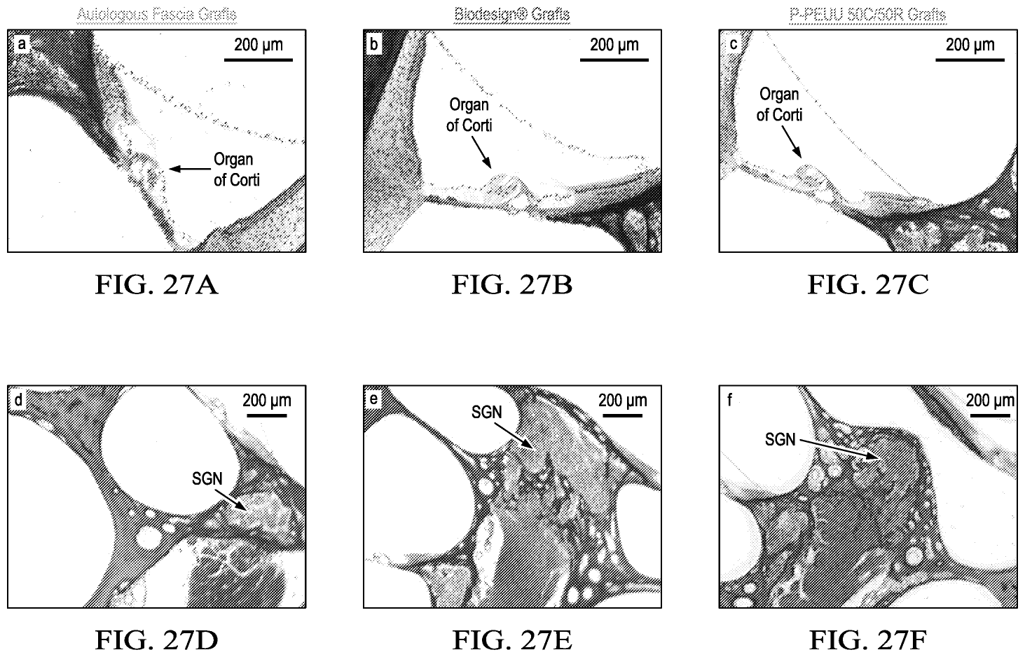

Additionally, histological sectioning of the cochlea 3 months following tympanoplasty with all three graft types show an intact organ of Corti and a healthy, normal population of SGN (FIGS. 27A-27F). Therefore, there is no reason to currently believe that the small molecule degradation products from either the control materials or P-PEUU were ototoxic. Indeed, the DPOAE and ABR results were similar to each other across the frequencies tested for P-PEUU grafts, suggesting a lack of ototoxicity. Due to the lack of ototoxicity observed in the cochlea following implantation with P-PEUU grafts and control materials, it is likely that the hearing thresholds changes seen by 3 months result from conductive hearing changes, not sensorineural hearing loss. During the surgical procedure, the ossicular chain is found to be fractured in several cases, which would indicate a conductive hearing loss stemming from the ossicular chain rather than the TM.

### Example 3

### Kit

Hearing impairment is a worldwide issue that significantly reduces quality of life. Service-members are particularly susceptible to short- and long-term hearing loss due to exposure to hazardous noise conditions from a variety of sources such as weapons training, artillery, aircraft, manufacturing, construction, or maintenance activities. A major concern for military health professionals is otologic injury caused by blast overpressure waves. The auditory system is the most vulnerable part of the body with regard to extreme air pressures and therefore is frequently damaged following blast exposure.

Blast-related casualties have increased over time, leading to significant otologic trauma in service-members. In the case of the active warfighter, impaired auditory performance is of high concern due to reduced situational awareness and operational readiness. The decreased ability to identify and locate sounds, communicate in loud environments, and control one's own noise production, among other disadvantages, can lead to dangerous working conditions, prolonged return to duty, and the inability to continue within an occupational specialty. Therefore, it is critical to ensure the timely and effective restoration of hearing to injured and active service-members.

In one instance, provided herein is a kit containing biomimetic poly(ester urethane urea) (PEUU) tissue engineering scaffolds for repair of tympanic membrane perforations at level III, and potentially level II, of military care. The kit will contain a series of synthetic scaffolds designed to reconstruct a wide range of perforation sizes and configurations. Each scaffold will have a unique radial and circumferential pattern that guides the regeneration of the native tympanic membrane architecture in the specific location of rupture. Facile, rapid, and effective perforation coverage will be achieved using a few standard otologic instruments through a novel bilayer graft design. Perforation closure using the novel graft can be performed using only local anesthesia with an approximate procedure time of 15 minutes (FIGS. 31 and 32). Biodegradability and biocompatibility of the PEUU material will ensure favorable patient outcomes with minimal post-operative care required. The kit will allow military personnel access to treatment for tympanic membrane perforations without the need for an operating room, thereby returning service members to duty faster while also providing improved restoration of auditory performance compared to current methods.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A melt-extrudable biodegradable ink for 3D printing, the ink comprising:
a soft segment block, and
a hard segment block,
wherein the molar ratio of soft segment block to hard segment block is in a range from 1:1.2 to 1:2.0.

2. The melt-extrudable biodegradable ink for 3D printing of claim 1, wherein the soft segment block comprises one or more of polycaprolactone (PCL), poly(ethylene glycol) (PEG), poly(hexamethylene carbonate) (PHC), poly(ethylene oxide) (PEO), poly(propylene oxide) (PPO), polylactide, (PLA), polyglycolide (PGA), poly(hydroxybutyrate) (P3HB and P4HB), poly(citric acid), poly(sebacic acid), amino acids, or other poly(ester), poly(ether), poly(carbonate), poly (tetramethylene oxide) (PTMO), poly(propylene fumarate) (PPF), and poly(amide) soft segments.

3. The melt-extrudable biodegradable ink for 3D printing of claim 2, wherein the soft segment block is a diol formed from one or more of polycaprolactone (PCL), poly(ethylene glycol) (PEG), poly(hexamethylene carbonate) (PHC), poly(ethylene oxide) (PEO), poly(propylene oxide) (PPO), polylactide, (PLA), polyglycolide (PGA), poly(hydroxybutyrate) (P3HB and P4HB), poly(citric acid), poly(sebacic acid), amino acids, or other poly(ester), poly(ether), poly(carbonate), poly (tetramethylene oxide) (PTMO), poly(propylene fumarate) (PPF), and poly(amide) soft segments.

4. The melt-extrudable biodegradable ink for 3D printing of claim 1, wherein the hard segment block comprises one or more of isophorene diisocyanate (IPDI), methyl diphenyl diisocyanate (MDI), 1-lysine diisocyanate (LDI), 1,4-butane diisocyanate (BDI), hexamethylene diisocyanate (HDI), trimethylhexamethylene diisocyanate (TMDI), ethyl diisocyanate (ELDI), methyl diisocyanate (MLDI), and 1,4-cyclohexane diisocyanate (CHDI).

5-6. (canceled)

7. The melt-extrudable biodegradable ink for 3D printing of claim 1, wherein the soft segment and hard segment are present in a ratio needed to make a poly(ester urethane)urea (PEUU).

8. The melt-extrudable biodegradable ink for 3D printing of claim 1, further comprising one or more chain extenders selected from the group consisting of: ethylene glycol, 1,4-butanediol, 1,4-cyclohexanedimethanol, 1,2-ethanediamine, 1,4-butanediamine, 2-amino-1-butanol, and 2-hydroxyethyl-2-hydroxyproponoate.

9. (canceled)

10. The melt-extrudable biodegradable ink for 3D printing of claim 1, further comprising a fugitive porogen.

11. The melt-extrudable biodegradable ink for 3D printing of claim 10, wherein the fugitive porogen is an oligomer including poly(ethylene glycol) or poly(propylene glycol).

12. The melt-extrudable biodegradable ink for 3D printing of claim 10, wherein the fugitive porogen comprises one or more of pluronic, alginate, gelatin, polyacrylic acid, poly(acrylate), poly(methacrylate), poly(maleic acid), poly(ethylene oxide), acrylates, methacrylates, water-soluble salts, water-soluble proteins, water-soluble small molecules, sugars, and water-soluble polysaccharides.

13. A melt-extrudable biodegradable ink for 3D printing, the ink comprising:
a biodegradable polymer, and
a fugitive porogen material,
wherein the fugitive porogen material is present at a weight percent of no more than 50 wt %.

14. The melt-extrudable biodegradable ink for 3D printing of claim 13, wherein the biodegradable polymer comprises a soft segment block and a hard segment block, and
wherein the soft segment block comprises one or more of polycaprolactone (PCL), poly(ethylene glycol) (PEG), poly(hexamethylene carbonate) (PHC), poly(ethylene oxide) (PEO), poly(propylene oxide) (PPO), polylactide, (PLA), polyglycolide (PGA), poly(hydroxybutyrate) (P3HB and P4HB), poly(citric acid), poly(sebacic acid), amino acids, or other poly(ester), poly(ether), poly(carbonate), poly (tetramethylene oxide) (PTMO), poly(propylene fumarate) (PPF), and poly(amide) soft segments, and/or
wherein the hard segment block comprises one or more of isophorene diisocyanate (IPDI), methyl diphenyl diisocyanate (MDI), 1-lysine diisocyanate (LDI), 1,4-butane diisocyanate (BDI), hexamethylene diisocyanate (HDI), trimethylhexamethylene diisocyanate (TMDI), ethyl diisocyanate (ELDI), methyl diisocyanate (MLDI), and 1,4-cyclohexane diisocyanate (CHDI).

15-16. (canceled)

17. The melt-extrudable biodegradable ink for 3D printing of claim 13, wherein the biodegradable polymer comprises one or more of hyaluronic acid (HA), poly(glycerol sebacate), poly(l,8-octanediol citrate), poly(limonene thioether), poly (lactic-co-glycolic acid) (PLGA), polyurethane, poly(ester urethane)urea (PEUU), poly(carbonate urethane) urea (PECUU), collagen, fibrin, nylon, and silk.

18. The melt-extrudable biodegradable ink for 3D printing of claim 13, wherein the fugitive porogen is an oligomer including poly(ethylene glycol) or poly(propylene glycol).

19. The melt-extrudable biodegradable ink for 3D printing of claim 13, wherein the fugitive porogen comprises one or more of pluronic, alginate, gelatin, polyacrylic acid, poly(acrylate), poly(methacrylate), poly(maleic acid), poly(ethylene oxide), acrylates, methacrylates, water-soluble salts, water-soluble proteins, water-soluble small molecules, sugars, and water-soluble polysaccharides.

20. A graft, comprising the melt-extrudable biodegradable ink for 3D printing of claim 13.

21. A method of fabricating the graft of claim 20, the method comprising melt-extruding the melt-extrudable biodegradable ink for 3D printing through a nozzle from a heated extrusion print head.

22-23. (canceled)

24. A method of implanting the graft of claim 20 into a patient to heal or augment a tympanic membrane or to replace a missing tympanic membrane or missing portion thereof, the method comprising:
accessing the damaged or missing tympanic membrane;
obtaining an appropriately sized and configured graft in the form of an artificial tympanic membrane device; and
securing the artificial tympanic membrane device to seal the a damaged portion of the tympanic membrane or to replace the missing tympanic membrane or missing portion thereof.

25. The method of claim 24, wherein the graft further comprises a cellular adhesion and/or a cell invasion-inducing material to promote tissue adhesion and cell growth.

26. The method of claim 24, wherein the graft promotes cellular alignment and deposition of extracellular matrix proteins along the print path via anisotropic topographical, chemical, or mechanical properties present in the graft.

27. A method of implanting the graft of claim 20 into a patient to heal or augment vasculature tissue, cartilage, a nerve conduit, a tendon, muscle tissue, or a bone or to replace a missing portion of vasculature tissue, cartilage, nerve conduit, tendon, muscle tissue, or bone, the method comprising:
accessing the damaged or missing vasculature tissue, cartilage, nerve conduit, tendon, muscle tissue, or bone;
obtaining an appropriately sized and configured graft in the form of an artificial vasculature tissue, cartilage, nerve conduit, tendon, muscle tissue, or bone device; and
securing the artificial cartilage, nerve conduit, tendon, muscle tissue, or bone device to seal a damaged portion of the vasculature tissue, cartilage, nerve conduit, tendon, muscle tissue, or bone, or to replace the missing portion of cartilage, nerve conduit, tendon, muscle tissue, or bone.
